SEQUENCE LISTING (39 sequences)

SEQ ID NO: 1 - DNA, 5860 bp, synthetic construct
  source 1..5860: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggatccgccaccatggccagcaaggtgtacgaccccgagcagcggaagcggatgatcaccggccctcagtggtgggctcggtgcaagcagatgaacgtgctggacagcttcatcaactactacgacagcgagaagcacgccgagaacgccgtgatcttcctgcacggcaacgccaccagcagctacctgtggcggcacgtggtgccccacatcgagcctgtggccagatgcatcatccccgacctgatcggcatgggcaagagcggcaagtccggcaacggcagctaccggctgctggaccactacaagtacctgaccgcttggtttgagctgctgaacctgcccaagaagatcatcttcgtcggccacgactggggcagcgccctggcctttcactacgcctacgagcaccaggaccggatcaaggccatcgtgcacatggaaagcgtggtggacgtgatcgagagctggatgggctggcccgacatcgaggaagaactggccctgatcaagagcgaagagggcgagaagatggtgctggaaaacaacttcttcgtggaaaccctgctgcccagcaagatcatgcggaagctggaacccgaagagttcgccgcctacctggaacccttcaaagaaaagggcgaagtgcggaggcccaccctgagctggcccagagagatccccctggtcaagggcggcaagcccgacgtggtgcagatcgtgcggaactacaacgcctacctgcgggccagcgacgacctgcctaagctgttcatcgagagcgaccccggcttcttcagcaacgccatcgtggaaggcgccaagaagttccccaacaccgagttcgtgaaagtgaagggcctgcacttcctccaggaagatgcccccgacgagatgggcaagtacatcaagagcttcgtggaacgggtgctgaagaacgagcagtgagcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 2 - DNA, 50 bp, Homo sapiens
  source 1..50: mol_type = other DNA
acatttgcttctgacacaactgtgttcactagcaacctcaaacagacacc

SEQ ID NO: 3 - DNA, 284 bp, synthetic construct
  source 1..284: mol_type = other DNA
gttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacag

SEQ ID NO: 4 - DNA, 6227 bp, synthetic construct
  source 1..6227: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgtcagagcgttgtcaccaccatccgctctgccctatctcgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggatccacatttgcttctgacacaactgtgttcactagcaacctcaaacagacaccatggccagcaaggtgtacgaccccgagcagcggaagcggatgatcaccggccctcagtggtgggctcggtgcaagcagatgaacgtgctggacagcttcatcaactactacgacagcgagaagcacgccgagaacgccgtgatcttcctgcacggcaacgccaccagcagctacctgtggcggcacgtggtgccccacatcgagcctgtggccagatgcatcatcccagatctgataggttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacaggtatgggcaagagcggcaagtccggcaacggcagctaccggcttctagaccactacaagtacctgaccgcttggtttgagctgctgaacctgcccaagaagatcatcttcgtcggccacgactggggcagcgccctggcctttcactacgcctacgagcaccaggaccggatcaaggccatcgtgcacatggaaagcgtggtggacgtgatcgagagctggatgggctggcccgacatcgaggaagaactggccctgatcaagagcgaagagggcgagaagatggtgctggaaaacaacttcttcgtggaaaccctgctgcccagcaagatcatgcggaagctggaacccgaagagttcgccgcctacctggaacccttcaaagaaaagggcgaagtgcggaggcccaccctgagctggcccagagagatccccctggtcaagggcggcaagcccgacgtggtgcagatcgtgcggaactacaacgcctacctgcgggccagcgacgacctgcctaagctgttcatcgagagcgaccccggcttcttcagcaacgccatcgtggaaggcgccaagaagttccccaacaccgagttcgtgaaagtgaagggcctgcacttcctccaggaagatgcccccgacgagatgggcaagtacatcaagagcttcgtggaacgggtgctgaagaacgagcagtgagcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 5 - DNA, 284 bp, synthetic construct
  source 1..284: mol_type = other DNA
gttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccagag

SEQ ID NO: 6 - DNA, 6227 bp, synthetic construct
  source 1..6227: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgtcagagcgttgtcaccaccatccgctctgccctatctcgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggatccacatttgcttctgacacaactgtgttcactagcaacctcaaacagacaccatggccagcaaggtgtacgaccccgagcagcggaagcggatgatcaccggccctcagtggtgggctcggtgcaagcagatgaacgtgctggacagcttcatcaactactacgacagcgagaagcacgccgagaacgccgtgatcttcctgcacggcaacgccaccagcagctacctgtggcggcacgtggtgccccacatcgagcctgtggccagatgcatcatcccagatctgataggttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccagaggtatgggcaagagcggcaagtccggcaacggcagctaccggcttctagaccactacaagtacctgaccgcttggtttgagctgctgaacctgcccaagaagatcatcttcgtcggccacgactggggcagcgccctggcctttcactacgcctacgagcaccaggaccggatcaaggccatcgtgcacatggaaagcgtggtggacgtgatcgagagctggatgggctggcccgacatcgaggaagaactggccctgatcaagagcgaagagggcgagaagatggtgctggaaaacaacttcttcgtggaaaccctgctgcccagcaagatcatgcggaagctggaacccgaagagttcgccgcctacctggaacccttcaaagaaaagggcgaagtgcggaggcccaccctgagctggcccagagagatccccctggtcaagggcggcaagcccgacgtggtgcagatcgtgcggaactacaacgcctacctgcgggccagcgacgacctgcctaagctgttcatcgagagcgaccccggcttcttcagcaacgccatcgtggaaggcgccaagaagttccccaacaccgagttcgtgaaagtgaagggcctgcacttcctccaggaagatgcccccgacgagatgggcaagtacatcaagagcttcgtggaacgggtgctgaagaacgagcagtgagcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 7 - DNA, 6188 bp, synthetic construct
  source 1..6188: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggatccacatttgcttctgacacaactgtgttcactagcaacctcaaacagacaccatggccagcaaggtgtacgaccccgagcagcggaagcggatgatcaccggccctcagtggtgggctcggtgcaagcagatgaacgtgctggacagcttcatcaactactacgacagcgagaagcacgccgagaacgccgtgatcttcctgcacggcaacgccaccagcagctacctgtggcggcacgtggtgccccacatcgagcctgtggccagatgcatcatcccagatctgatcggcatgggcaagagcggcaaggttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccacagtccggcaacggcagctaccggcttctagaccactacaagtacctgaccgcttggtttgagctgctgaacctgcccaagaagatcatcttcgtcggccacgactggggcagcgccctggcctttcactacgcctacgagcaccaggaccggatcaaggccatcgtgcacatggaaagcgtggtggacgtgatcgagagctggatgggctggcccgacatcgaggaagaactggccctgatcaagagcgaagagggcgagaagatggtgctggaaaacaacttcttcgtggaaaccctgctgcccagcaagatcatgcggaagctggaacccgaagagttcgccgcctacctggaacccttcaaagaaaagggcgaagtgcggaggcccaccctgagctggcccagagagatccccctggtcaagggcggcaagcccgacgtggtgcagatcgtgcggaactacaacgcctacctgcgggccagcgacgacctgcctaagctgttcatcgagagcgaccccggcttcttcagcaacgccatcgtggaaggcgccaagaagttccccaacaccgagttcgtgaaagtgaagggcctgcacttcctccaggaagatgcccccgacgagatgggcaagtacatcaagagcttcgtggaacgggtgctgaagaacgagcagtgagcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 8 - DNA, 6188 bp, synthetic construct
  source 1..6188: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggatccacatttgcttctgacacaactgtgttcactagcaacctcaaacagacaccatggccagcaaggtgtacgaccccgagcagcggaagcggatgatcaccggccctcagtggtgggctcggtgcaagcagatgaacgtgctggacagcttcatcaactactacgacagcgagaagcacgccgagaacgccgtgatcttcctgcacggcaacgccaccagcagctacctgtggcggcacgtggtgccccacatcgagcctgtggccagatgcatcatcccagatctgatcggcatgggcaagagcggcaaggttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgtctttctgtttgtccccagagtccggcaacggcagctaccggcttctagaccactacaagtacctgaccgcttggtttgagctgctgaacctgcccaagaagatcatcttcgtcggccacgactggggcagcgccctggcctttcactacgcctacgagcaccaggaccggatcaaggccatcgtgcacatggaaagcgtggtggacgtgatcgagagctggatgggctggcccgacatcgaggaagaactggccctgatcaagagcgaagagggcgagaagatggtgctggaaaacaacttcttcgtggaaaccctgctgcccagcaagatcatgcggaagctggaacccgaagagttcgccgcctacctggaacccttcaaagaaaagggcgaagtgcggaggcccaccctgagctggcccagagagatccccctggtcaagggcggcaagcccgacgtggtgcagatcgtgcggaactacaacgcctacctgcgggccagcgacgacctgcctaagctgttcatcgagagcgaccccggcttcttcagcaacgccatcgtggaaggcgccaagaagttccccaacaccgagttcgtgaaagtgaagggcctgcacttcctccaggaagatgcccccgacgagatgggcaagtacatcaagagcttcgtggaacgggtgctgaagaacgagcagtgagcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 9 - DNA, 284 bp, synthetic construct
  source 1..284: mol_type = other DNA
gttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctgcctgtgttccttgtgtctttctgtttgtccccacag

SEQ ID NO: 10 - DNA, 6188 bp, synthetic construct
  source 1..6188: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactataggggatccacatttgcttctgacacaactgtgttcactagcaacctcaaacagacaccatggccagcaaggtgtacgaccccgagcagcggaagcggatgatcaccggccctcagtggtgggctcggtgcaagcagatgaacgtgctggacagcttcatcaactactacgacagcgagaagcacgccgagaacgccgtgatcttcctgcacggcaacgccaccagcagctacctgtggcggcacgtggtgccccacatcgagcctgtggccagatgcatcatcccagatctgatcggcatgggcaagagcggcaaggttggtatcaaggttacaagacaggtttaaggagaccaatagaaactgggcatgtggagacagagaagactcttgggtttctgagagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctgcctgtgttccttgtgtctttctgtttgtccccacagtccggcaacggcagctaccggcttctagaccactacaagtacctgaccgcttggtttgagctgctgaacctgcccaagaagatcatcttcgtcggccacgactggggcagcgccctggcctttcactacgcctacgagcaccaggaccggatcaaggccatcgtgcacatggaaagcgtggtggacgtgatcgagagctggatgggctggcccgacatcgaggaagaactggccctgatcaagagcgaagagggcgagaagatggtgctggaaaacaacttcttcgtggaaaccctgctgcccagcaagatcatgcggaagctggaacccgaagagttcgccgcctacctggaacccttcaaagaaaagggcgaagtgcggaggcccaccctgagctggcccagagagatccccctggtcaagggcggcaagcccgacgtggtgcagatcgtgcggaactacaacgcctacctgcgggccagcgacgacctgcctaagctgttcatcgagagcgaccccggcttcttcagcaacgccatcgtggaaggcgccaagaagttccccaacaccgagttcgtgaaagtgaagggcctgcacttcctccaggaagatgcccccgacgagatgggcaagtacatcaagagcttcgtggaacgggtgctgaagaacgagcagtgagcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 11 - RNA, 60 nt, synthetic construct
  source 1..60: mol_type = other RNA
agagcctctaattgttccttgtgtctttctgtttgtccccagagtccccatggaaaagcc

SEQ ID NO: 12 - RNA, 40 nt, synthetic construct
  source 1..40: mol_type = other RNA
acagaaagacgcccccttattcgtccccgcctggggacaa

SEQ ID NO: 13 - RNA, 40 nt, synthetic construct
  source 1..40: mol_type = other RNA
acagaaagaccgcccccttattcgtccccgctggggacaa

SEQ ID NO: 14 - RNA, 40 nt, synthetic construct
  source 1..40: mol_type = other RNA
aaacagaaagcccccttattcgtcccccagctctggggac

SEQ ID NO: 15 - RNA, 54 nt, synthetic construct
  source 1..54: mol_type = other RNA
agaaagacacaatctctgcctagcccccttattcgtccccgcctggggacaaac

SEQ ID NO: 16 - RNA, 54 nt, synthetic construct
  source 1..54: mol_type = other RNA
agaaagacacaatctctgcctacgcccccttattcgtccccgctggggacaaac

SEQ ID NO: 17 - RNA, 54 nt, synthetic construct
  source 1..54: mol_type = other RNA
acagaaagacactctctgcctacccccttattcgtcccccagctctggggacaa

SEQ ID NO: 18 - RNA, 40 nt, synthetic construct
  source 1..40: mol_type = other RNA
gacaaacagaaagacgcccccttattcgtccccgcctggg

SEQ ID NO: 19 - RNA, 54 nt, synthetic construct
  source 1..54: mol_type = other RNA
gacaaacagaaagacacaatctctgcctagcccccttattcgtccccgcctggg

SEQ ID NO: 20 - RNA, 41 nt, synthetic construct
  source 1..41: mol_type = other RNA
gctcgtcacaggcccccttattcgtccccgctagcagcgat

SEQ ID NO: 21 - DNA, 70 bp, synthetic construct
  source 1..70: mol_type = other DNA
acaaacagaaagacacaaggtctctgcctagcccccttattcgtcctccccttttccctggggactgtgg

SEQ ID NO: 22 - DNA, 54 bp, synthetic construct
  source 1..54: mol_type = other DNA
gtgccgtatgcatctctgcctagcccccttattcgtcctcccacaacttgcttg

SEQ ID NO: 23 - DNA, 189 bp, synthetic construct
  source 1..189: mol_type = other DNA
actagtatatttgcatgtcgctatgtgttctgggaaatcaccataaacgtgaaatgtctttggatttgggaatcttataagttctgtatgagaccactctttcccacaaacagaaagacacaaggtctctgcctagcccccttattcgtcctccccttttccctggggactgtggttttttgcggccgc

SEQ ID NO: 24 - DNA, 173 bp, synthetic construct
  source 1..173: mol_type = other DNA
actagtatatttgcatgtcgctatgtgttctgggaaatcaccataaacgtgaaatgtctttggatttgggaatcttataagttctgtatgagaccactctttcccgtgccgtatgcatctctgcctagcccccttattcgtcctcccacaacttgcttgttttttgcggccgc

SEQ ID NO: 25 - DNA, 4460 bp, synthetic construct
  source 1..4460: mol_type = other DNA
actagtatatttgcatgtcgctatgtgttctgggaaatcaccataaacgtgaaatgtctttggatttgggaatcttataagttctgtatgagaccactctttcccacaaacagaaagacacaaggtctctgcctagcccccttattcgtcctccccttttccctggggactgtggttttttgcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 26 - DNA, 4444 bp, synthetic construct
  source 1..4444: mol_type = other DNA
actagtatatttgcatgtcgctatgtgttctgggaaatcaccataaacgtgaaatgtctttggatttgggaatcttataagttctgtatgagaccactctttcccgtgccgtatgcatctctgcctagcccccttattcgtcctcccacaacttgcttgttttttgcggccgcaaaatcagcctcgactgtgccttctagttgccagccatctgttgtttgcccctcccccgtgccttccttgaccctggaaggtgccactcccactgtcctttcctaataaaatgaggaaattgcatcacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcattaccaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatcatttagaataaattttgtgtcgcccttaattgtgagcggataacaattacgagcttcatgcacagtggcgttgacattgattattgactagcatgttctttcctgcgttatcccctgattctgtggataaccgtattaccgccatgcattagttattaataacatacgctctccatcaaaacaaaacgaaacaaaacaaactagcaaaataggctgtccccagtgcaagtgcaggtgccagaacatttctct

SEQ ID NO: 27 - DNA, 1020 bp, Homo sapiens
  source 1..1020: mol_type = genomic DNA
ggaacttgcagagctaggggttcagaggggtgaagaagcatgtttcagttctgccttttaaatgatcccaaaaaggttagcagttttcaaatgacatttgcagacagcctcatttaattccatgagaagggtgagcaaaggattatcttgttgaaactgattcctggagagactgagcaccgtacctgagttcaaacttgggaatgttctagatggtgactcaggcccaggcaccaaccagcagaatgggcctcagcctgacaacccttctgtaccaggcctgactctttggttgctgaactttggagaggcctgggggggtcagcggcaggcagacgagtgagtggctttggtgacaggtcctcaggggcagccaggcagtgtgactctcgttcaatagtaacgtttgtcagagcgttgtcaccaccatccgctctgccctatctctgacattgctatggagagcctctaattgttccttgtgcccccttattcgtccccgcagtccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcagg

SEQ ID NO: 28 - DNA, 20 bp, Homo sapiens
  source 1..20: mol_type = genomic DNA
aatgttctagatggtgactc

SEQ ID NO: 29 - DNA, 20 bp, Homo sapiens
  source 1..20: mol_type = genomic DNA
ggtgactcaggcccaggcac

SEQ ID NO: 30 - DNA, 20 bp, Homo sapiens
  source 1..20: mol_type = genomic DNA
tcaggcccaggcaccaacca

SEQ ID NO: 31 - DNA, 20 bp, Homo sapiens
  source 1..20: mol_type = genomic DNA
gtccccgcagtccccaggga

SEQ ID NO: 32 - DNA, 20 bp, Homo sapiens
  source 1..20: mol_type = genomic DNA
cagtccccagggaaaagcct

SEQ ID NO: 33 - DNA, 20 bp, Homo sapiens
  source 1..20: mol_type = genomic DNA
cagggaaaagcctttcgctc

SEQ ID NO: 34 - DNA, 20 bp, Homo sapiens
  source 1..20: mol_type = genomic DNA
cgctctaaagtggagttgat

SEQ ID NO: 35 - DNA, 20 bp, Homo sapiens
  source 1..20: mol_type = genomic DNA
taaagtggagttgattgcgt

SEQ ID NO: 36 - DNA, 20 bp, Homo sapiens
  source 1..20: mol_type = genomic DNA
atccacccaggtcatggtga

SEQ ID NO: 37 - DNA, 20 bp, Homo sapiens
  source 1..20: mol_type = genomic DNA
gccccggcaggaagcgaaaa

SEQ ID NO: 38 - DNA, 20 bp, Homo sapiens
  source 1..20: mol_type = genomic DNA
cggcaggaagcgaaaagctg

SEQ ID NO: 39 - DNA, 20 bp, Homo sapiens
  source 1..20: mol_type = genomic DNA
aagcgaaaagctgaggccga

(19) United States
(12) Patent Application Publication
(10) Pub. No.: US 2024/0417728 A1
(43) Pub. Date: Dec. 19, 2024

(54) FOLDING OLIGONUCLEOTIDES

(71) Applicant: RNA MORPH LTD, Even Yehuda (IL)

(72) Inventor: Eitan LEV, Even Yehuda (IL)

(73) Assignee: RNA MORPH LTD, Even Yehuda (IL)

(21) Appl. No.: 18/727,281

(22) PCT Filed: Sep. 21, 2023
(86) PCT No.: PCT/IL2023/051028

§ 371 Date: Jul. 8, 2024

Related U.S. Application Data
(60) Provisional application No. 63/376,622, filed on Sep. 22, 2022.

(51) Int. Cl. C12N 15/113

(52) U.S. Cl. CPC C12N 15/113; C12N 2310/11

(57) ABSTRACT

The invention provides folding oligonucleotides and uses thereof to rectify genetic mutations in a target RNA molecule, or to attach specific motifs to the target RNA molecule.

### TECHNOLOGICAL FIELD

The present disclosure is in the field of engineering nucleic acid-based therapeutics.

### BACKGROUND

In the natural transcription process that takes place in the cell nucleus RNA (ribonucleic acid) is transcribed from a DNA (deoxyribonucleic acid) template. During this process a pre-mRNA (pre-messenger RNA) transcript is formed. The pre-mRNA becomes mature mRNA after processing. RNA processing includes 5′ capping, 3′ polyadenylation, and splicing, including alternative splicing. The splicing process removes all the introns (non-coding regions of RNA) and splices back together the exons (the coding regions).

Splicing of pre-mRNA occurs at consensus sequences near the 5′ and 3′ ends of introns, known as 5′(the donor) and 3′ (the acceptor) splice-sites (5′ss and 3′ss) by a large, dynamic RNA-protein complex called the spliceosome (Nelson K. K., and Green M. R. Genes Dev 1989:3:1562-1571). The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), termed the polypyrimidine tract (PTT). The consensus sequence for an intron (in IUPAC nucleic acid annotation) is G-G-[cut]-G-U-R-A-G-U (donor site) . . . intron sequence . . . Y-U-R-A-C (branch sequence 20-50 nucleotides upstream of acceptor site) . . . Y-rich-N-C-A-G-[cut]-G (acceptor site). The branch point sequence is a cis-acting intronic motif required for mRNA splicing.

Mutations in 5′ss, 3′ss, BP sequence and the polypyrimidine tract (PPT) cause genetic diseases by altered splicing efficiency (Faustino N. A., and Cooper T. A. Genes Dev. 2003:17:419-437). Cryptic 5′ss or 3′ss instead of canonical splice-sites are sometimes activated (Buratti E., et al. Nucleic Acids Res. 2011:39:D86-D91).

Eukaryotic genomes contain “authentic” splice sites (which are present in the wildtype pre-mRNA) as well as large numbers of cryptic splice sites (css), which are generally held to be dormant (or undetectably used) sites unless activated by mutation of a nearby authentic splice site. Namely, point mutations in the underlying DNA or errors during transcription can activate a cryptic splice site in part of the transcript that usually is not spliced. This results in a mature mRNA with a missing section of an exon, which can manifest as a deletion or truncation in the final protein, or an added sequence to the exon that disrupts the reading frame and results in a different protein sequence or truncated protein sequence due to inclusion of stop codons.

Trans-splicing is a splicing reaction ligating two exons from two different RNA molecules. This mechanism occurs naturally in eukaryotic cells, including human cells.

A review article by Berger et al., (2016 WIREs RNA, 7:487-498) describes spliceosome-mediated RNA Trans-splicing (SMaRT) as a strategy to design gene therapy solutions for genetic diseases. SMaRT relies on the correction of mutations at the post-transcriptional level by modifying the mRNA sequence. To achieve this, an exogenous RNA is introduced into the target cell, usually by means of gene transfer, to induce a splice event in trans between the exogenous RNA and the target endogenous pre-mRNA.

This produces a chimeric mRNA composed partly of exons of the latter, and partly of exons of the former, encoding a sequence free of mutations. The principal challenge of SMaRT technology is to achieve a reaction as complete as possible, i.e., resulting in 100% repairing of the endogenous mRNA target.

### GENERAL DESCRIPTION

In one aspect, the present invention provides an oligonucleotide, comprising from 5′ to 3′:
A first sequence of nucleic acids that is complementary in its 3′ to 5′ direction to a region in a pre-mRNA or mRNA target molecule;
A second sequence of nucleic acids comprising a heterologous sequence; and
A third sequence of nucleic acids that is complementary in its 3′ to 5′ direction to a sequence of nucleic acids in the pre mRNA or mRNA target molecule which is positioned upstream to the hybridization site of the first sequence of nucleic acids;
and wherein said first and third sequences of nucleic acids hybridize to the same intron, or to the same exon, or to successive intron and exon, or to successive exon and intron.

In one embodiment, said heterologous sequence comprises a sequence which is identical to and is in the same 5′->3′ direction as the sequence of an exon, an intron, a splice site, a 5′ UTR, a 3′ UTR, or a fragment or portion thereof of the wildtype pre-mRNA or mRNA target molecules.

In one embodiment, said heterologous sequence encodes a portion of an exon.

In one embodiment, said oligonucleotide is an antisense oligonucleotide.

In one embodiment, said oligonucleotide is synthesized as a linear single stranded molecule and forms an open circle structure upon hybridization with the pre-mRNA target molecule.

In one embodiment, hybridization of the oligonucleotide with the target pre-mRNA or mRNA molecule masks a mutation in the pre-mRNA or mRNA molecule and aligns the second sequence of nucleic acids such that the mutated sequence of the pre-mRNA is replaced with the sequence of the wildtype pre-mRNA, thereby allowing the translation of a functional protein.

In one embodiment, hybridization of the oligonucleotide with the target pre-mRNA or mRNA molecule introduces to the endogenous pre-mRNA or mRNA molecules a heterologous motif.

In one embodiment, said second sequence of nucleic acids binds to a cellular complex.

In one embodiment, said nucleic acids are ribonucleotides.

In one embodiment, said mutated site comprises a single base mutation, a substitution, a deletion mutation, an insertion mutation, or an InDel mutation.

In one embodiment, said second sequence of nucleic acids comprises (i) a portion of an intron ending with an acceptor site; (ii) a heterologous sequence to be trans-spliced into the target pre-mRNA molecule; (iii) a portion of an intron comprising a donor site, and optionally a branch point and a PPT sequence, ending at the proximity of an acceptor site sequence in the wildtype exon.

In some embodiments, said oligonucleotide is selected from a group consisting of Ocirc 1 (SEQ ID NO: 12), Ocirc 2 (SEQ ID NO: 13), Ocirc 3 (SEQ ID NO: 14), Ocirc 4 (SEQ ID NO: 15), Ocirc 5 (SEQ ID NO: 16), Ocirc 6 (SEQ ID NO: 17), Ocirc 7 (SEQ ID NO: 18), and Ocirc 8 (SEQ ID NO: 19).

In another aspect, the present invention provides an oligonucleotide, comprising from 5′ to 3′:

A first sequence of nucleic acids that is complementary in its 3′ to 5′ direction to a region in a pre-mRNA target molecule:
A second sequence comprising:
(i) a portion of an intron ending with an acceptor site;
(ii) a heterologous sequence to be trans-spliced into the target pre-mRNA molecule; and
(iii) a portion of an intron comprising a donor site, and optionally a branch point and a PPT sequence, ending at the proximity of an acceptor site sequence in the wildtype exon, and

A third sequence of nucleic acids that hybridizes in its 3′ to 5′ direction with a sequence of nucleic acids positioned upstream to the hybridization site of the first sequence of nucleic acids in said pre-mRNA target molecule preceding said full or partial acceptor site sequence.

In one embodiment, said heterologous sequence comprises a sequence which is identical to and is in the same 5′->3′ direction as the sequence of an exon, an intron, a splice site, or a fragment or portion thereof of the wildtype pre-mRNA molecule terminating in the YAG acceptor site following the second complementary sequence at the 3′ terminus of the oligonucleotide.

In one embodiment, said heterologous sequence encodes a portion of an exon.

In another embodiment, the present invention provides a delivery vector or an isolated cell comprising the oligonucleotide of the invention

In another aspect, the present invention provides a method for substitution of an endogenous nucleic acid sequence comprising bringing into contact the oligonucleotides, or the delivery vector of the invention with a target cell comprising said endogenous nucleic acid sequence.

In another aspect, the present invention provides a method of treating Rett syndrome, said method comprises administering the oligonucleotides, or the delivery vector, or the isolated cell of the invention, to a patient in need thereof.

In another aspect, the present invention provides the oligonucleotides, or the delivery vector, or the isolated cell of the invention, for use in a method of treating Rett syndrome.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 is a schematic illustration of a point mutation (a C to G mutation) between an exon and an intron in a pre-mRNA transcript. This point mutation alters the original splice location and invokes the use of a cryptic splice site.

FIG. 2 is an illustration of an open circle folding oligonucleotide according to an embodiment of the present disclosure. The folding oligonucleotide is shown in a compressed manner. The folding oligonucleotide hybridizes with and masks the original mutated site.

FIG. 3 is an illustration of an open circle folding oligonucleotide according to an embodiment of the present disclosure wherein the length of the second sequence of nucleic acids which is exposed upwards is identical to the accumulated lengths of the flaps.

FIG. 4 is an illustration of an open circle folding oligonucleotide according to an embodiment of the present disclosure wherein the length of the second sequence of nucleic acids which is exposed upwards is shorter than the accumulated lengths of the flaps thereby rectifying an insertion mutation.

FIG. 5 is an illustration of an open circle folding oligonucleotide according to an embodiment of the present disclosure wherein the length of the second sequence of nucleic acids which is exposed upwards is longer than the accumulated lengths of the flaps thereby rectifying a deletion mutation.

FIG. 6 is an illustration of an open circle folding oligonucleotide according to an embodiment of the present disclosure wherein the second sequence of nucleic acids which is exposed upwards comprises a heterologous motif, e.g., a sequence motif that serves as a recognition site for an RNA binding protein.

FIG. 7 is an illustration of an open circle folding oligonucleotide according to an embodiment of the present disclosure wherein the G mutation is masked with a hybridizing C.

FIG. 8 is an illustration of an open circle folding oligonucleotide according to an embodiment of the present disclosure wherein an inverted C nucleotide is placed at the end of the exposed part of the open circle folding oligonucleotide, aligned with the mutated G.

FIG. 9 is an illustration of an open circle folding oligonucleotide according to an embodiment of the present disclosure wherein the mutated GAG acceptor site sequence is masked, and a correct CAG sequence is exposed at the terminus of the exposed part of said open circle folding oligonucleotide

FIG. 10 is an illustration of a folding oligonucleotide according to an embodiment of the present disclosure wherein the folding oligonucleotide mediates a trans-splicing event, to fix a mutation in the acceptor site.

FIG. 11 is an illustration of a folding oligonucleotide according to an embodiment of the present disclosure wherein the folding oligonucleotide mediates a trans-splicing event, to fix a mutation in an exon.

FIG. 12 is an illustration of a folding oligonucleotide according to an embodiment of the present disclosure wherein the folding oligonucleotide mediates a trans-splicing event, to fix a mutation in the acceptor site.

FIG. 13 shows exemplary sequences of the folding oligonucleotide of the invention.

FIG. 14 shows results of a splicing simulation.

FIG. 15 is an illustration of the pCMV-Green Renilla Luc plasmid. The black arrow marks the insertion point of human beta globin 5′UTR (hBB) in the derivative plasmids. The hollow arrow generally marks the Green Renilla Luc gene in which additional changes were made in the derivative plasmids.

FIG. 16 is an illustration of the binding of a regular RNA oligonucleotide to the 3′ region of the 3^{rdd} intron of human MECP2 gene which forms an Ocirc structure upon binding.

FIG. 17 shows results of running the various samples on a polyacrylamide gel: lane 1—Ocirc template alone, incubated at 70° C.; lane 2—Ocirc template alone, without incubation at 70° C.; lane 3—Ocirc template+Ocirc 1; lane 4—Ocirc 1 without a template, and without incubation at 70° C.; lane 5—Ocirc 1 without a template with incubation at 70° C.; Lane 6—RNA ladder; lane 7—Ocirc template alone, without incubation at 70° C.; lane 8—Ocirc template+Ocirc 4; lane 9—Ocirc 4 without a template and without incubation at 70° C.; lane 10—Ocirc 4 without a template with incubation at 70° C.; and Lane 11—RNA ladder. The numbers on the righthand side of the gel indicate the size of the mRNA ladder (no, of nucleotides).

FIG. 18 shows results of running the various samples on a polyacrylamide gel: lane 1—Ocirc template alone; lane 2—Ocirc 5; lane 3—Ocirc template+Ocirc 5; lane 4—RNA ladder; lane 5—Ocirc 6; lane 6—Ocirc template+Ocirc 6; lane 7—Ocirc 7; lane 8—Ocirc template+Ocirc 7; lane 9—Ocirc template alone; lane 10—Ocirc 8; lane 11—Ocirc template+Ocirc 8; lane 12—RNA ladder. The numbers on the righthand side of the gel indicate the size of the mRNA ladder.

FIGS. 19A and 19B show results of running the various samples on a polyacrylamide gel. FIG. 19A: lane 1—Ocirc template alone; lane 2—Ocirc 1; lane 3—Ocirc template+Ocirc 1; lane 4—Ocirc template+Ocirc 1+recombinant U2AF2 protein; lane 5—RNA ladder; lane 6—Ocirc 2; lane 7—Ocirc template+Ocirc 2; FIG. 19B: lane 1—Ocirc template alone; lane 2—Ocirc 3; lane 3—Ocirc template+Ocirc 3; lane 4—Ocirc template+Ocirc 3+recombinant U2AF2 protein; lane 5—RNA ladder; lane 6—Ocirc 4; lane 7—Ocirc template+Ocirc 4; lane 8—Ocirc template+Ocirc 4+recombinant U2AF2 protein; lane 9—Ocirc control; lane 10—Ocirc template+Ocirc control.

FIG. 20 is an illustration of the binding of pH1-Ocirc-AS to the 3′ region of the 3^{rd} intron of human MECP2 gene.

### DETAILED DESCRIPTION OF EMBODIMENTS

The present disclosure provides novel folding oligonucleotides.

As used herein the term “oligonucleotide” refers to a molecule that consists of several repeating units (i.e., monomers) of nucleic acids. In an embodiment, the oligonucleotide is a recombinant nucleic acid. In the context of the present invention the oligonucleotide is designed such that it is capable of folding and is referred to herein as a folding oligonucleotide.

As used herein the term “folding oligonucleotide” refers to a molecule comprising a sequence of nucleotides that is partially complementary to the nucleic acid sequence of a target RNA molecule, wherein upon binding to the target RNA the oligonucleotide folds in a manner that exposes a heterologous sequence of nucleic acids thus forming a chimeric molecule comprising endogenous and heterologous nucleic acid sequences.

As used herein the term “heterologous” refers to a sequence of nucleic acids that originates from a non-endogenous source, namely that is external to the cell in which it is expressed. The heterologous sequence may be identical with the wild-type sequence of the target mRNA thereby rectifying a mutation in the endogenous sequence, and/or it may comprise an external motif, e.g., a recognition site for an RNA binding protein, or any other desired sequence that may react with cell complexes.

The present disclosure thus provides methods for substitution of an original endogenous nucleic acid sequence with a new sequence using the folding oligonucleotides of the invention.

The novel folding oligonucleotides of the invention may be used for at least the following:
(i) Rectifying/substituting a genetic mutation in the target RNA. Accordingly, the folding oligonucleotides of the invention may mask a nucleic acid mutation in the target RNA and expose instead of the mutation, a corrected, non-mutated sequence (also referred to herein as a “wild-type” sequence), and by that allowing normal expression of the target gene; and/or
(ii) attaching an element to the target RNA. Accordingly, the folding oligonucleotides of the invention may attach to the target nucleic acid specific motifs that can serve as binding sites for various RNA binding proteins (RBP), to facilitate translation, splicing and/or silencing processes. For example, the folding oligonucleotides of the invention may bind to a wild-type sequence in the UTR and attract an RBP.

As used herein the term “RNA binding protein(s)” or “RBP” refers to proteins which contain RNA binding domains and bind to single stranded or double stranded RNA molecules via specific sequence motifs. Such sequence motifs are usually located in the untranslated regions (UTRs) of the transcript, but they are also present in the introns and exons, for instance splicing enhancers/suppressors. RBPs contain various structural elements, such as RNA recognition motifs (RRM), dsRNA binding domain, zinc fingers and others.

The RBP regulate most, if not all. RNA functions in gene expression including pre-mRNA splicing, mRNA trafficking (localization), RNA processing (e g., polyadenylation), modification, stability, silencing, and regulation of protein synthesis (translation) via formation of ribosomes, spliceosomes, and RNA-induced silencing complexes (RISC).

There are several thousands of genes encoding RBPs in humans, a list of RBPs can be found in the Eukaryotic RBP Database (EuRBPDB). While not limited thereto, the folding oligonucleotides of this disclosure may, therefore, be used for correcting genetic mutations by complexing with a pre-mRNA or mRNA molecule carrying a mutation such that the cell's translation mechanism will “read” a corrected mature mRNA sequence giving rise to the synthesis of a functional protein.

The methods and compounds of the present invention may be used to rectify mutations in one or both of an intron and an exon of the pre-mRNA molecule. Moreover, the methods and compounds of the present invention may also be used to rectify mutations in an exon of a mature mRNA molecule.

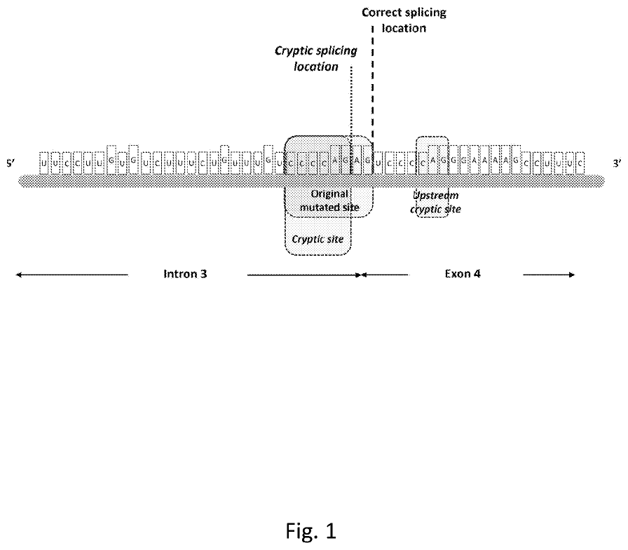

As used herein the term “mutation” refers to an insertion or deletion of one or more nucleotides, including Indel mutations, as well as to a substitution of one or more nucleotides. The term also encompasses point mutations in which a single wild-type nucleotide is substituted by another nucleotide (e.g., a C to G mutation as exemplified in FIG. 1).

In one aspect, the disclosure relates to compositions and methods for rectifying a mutant MCCP2 gene in a cell or a subject. McCP2 gene refers to methyl CpG binding protein 2 gene. The MCCP2 protein plays important roles (e.g., functions as a transcriptional repressor, or transcriptional activator) in nerve cells, such as mature neurons. One example of a MeCP2 gene is represented by GenBank Accession Number NM 001110792 (MeCP2-e1). Another example of a MeCP2 gene is represented by Genbank Accession Number NM_001110792 (MeCP2-e2).

Mutations in MeCP2 are the major cause of Rett syndrome, a neurodevelopmental disorder. Various types of mutations in the gene can cause the disease, including a mutation in the splicing site between the 3^{rd} intron and the fourth exon, a C to G mutation which abolishes the normal splicing site and causes a mis-splicing event.

Accordingly, in one aspect, the present invention provides a method of treating Rett syndrome, said method comprises administration of the folding oligonucleotides of the invention, or a vector comprising the folding oligonucleotides, to a patient in need thereof.

The folding oligonucleotides may be used to rectify any mutation in the MeCP2 gene, including but not limited to, the C to G point mutation which causes the mis-splicing event.

As used herein, “treating” Rett syndrome means administration to an individual by any suitable dosage regimen, procedure and/or administration route of a composition comprising the oligonucleotides of the present invention, with the object of achieving a desirable clinical/medical endpoint, including but not limited to, stopping, or slowing progression, reversing, or reducing symptoms of the disease.

### Open Circle Folding Oligonucleotide

The present disclosure concerns folding oligonucleotides and methods for compensating for a nucleic acid mutation in a target nucleic acid sequence using an oligonucleotide capable of folding into an open circle structure (also referred to herein as an Ocric oligonucleotide). The open circle structure formed by the folding oligonucleotides of the invention upon hybridizing with the target molecule, resembles the structure of circular RNA (circRNA), except for being open (not forming a closed circle). In some embodiments, the folding oligonucleotides of the invention are synthesized as circular RNA. It should be emphasized however that the assumed roles of circRNA in nature differ from the proposed uses of the folding oligonucleotides of the invention.

The folding oligonucleotides of the invention may be synthetically produced and administered to the cell using methods known in the art, or they may be natural RNA, at which case the oligonucleotide is produced for example by a gene/plasmid that is inserted into the cell nucleus.

In an embodiment, the folding oligonucleotides are antisense molecules (also referred to herein as “open circle antisense oligonucleotides (ASO)” or OcircASO), The folding oligonucleotide is generated as a linear single stranded molecule and upon interaction with the target sequence it folds into an open circle structure.

The folding oligonucleotide may be comprised of ribonucleotides, deoxyribonucleotides, nucleic acid analogues, or any combination thereof.

In one embodiment, the folding oligonucleotide comprises 3 parts from 5′ to 3′.

A first sequence of nucleic acids that that is complementary in its 3′ to 5′ direction to a sequence of nucleic acids in the pre mRNA or mRNA target molecule.

A second sequence of nucleic acids comprising a heterologous sequence, namely a sequence which mimics and is in the same 5′->3′ direction as the sequence of an exon, an intron, a splice site, a 5′ UTR, a 3′ UTR, or a fragment thereof of the wildtype pre-mRNA or mRNA target molecules; and

a third sequence of nucleic acids that is complementary in its 3′ to 5′ direction to a sequence of nucleic acids in the pre mRNA or mRNA target molecule which is positioned upstream to the hybridization site of the first sequence of nucleic acids.

As used herein the term “from 5” to 3′″ refers to the directionality or orientation of nucleotides of a single strand of DNA or RNA. The 5′ and 3′ specifically refer to the 5^{th} and the 3^{rd} carbon atoms in the deoxyribose/ribose sugar ring forming a 5′ end and a 3′ end.

The first and the third sequences of nucleic acids which hybridize with the target mRNA or pre-mRNA are also referred to herein as the “binding sites” or “flaps”.

In accordance with the invention, the first and third sequences of nucleic acids do not hybridize with successive introns, this is in clear contrast to the trans-splicing methods known in the art. The first and third sequences of nucleic acids will hybridize to the same intron, same exon, or to successive intron and exon or exon and intron.

In one embodiment, the first and the third nucleic acid sequences are designed and synthesized such that they would be complementary and therefore hybridize to a stretch of consecutive nucleic acids in the target molecule. Namely, the 3′ to 5′ sequence of the first and the third sequences is complementary to a 5′ to 3′ consecutive sequence of the target molecule.

In another embodiment, the first and the third nucleic acid sequences are designed and synthesized such that they would be complementary and therefore hybridize to a stretch of nucleic acids in the target molecule which is not sequential or consecutive. Namely, the hybridization sites of the first and the third nucleic acid sequences on the target pre-mRNA or RNA molecule are separated by a stretch of nucleic acids.

Since the first and the third sequences are complementary to a stretch of nucleic acids in the target being upstream one to the other (either consecutively or separated by a stretch of nucleic acids), upon hybridization of these sequences with the target molecule, the oligonucleotide of the invention folds on itself both at the 5′ and the 3′ termini. Due to steric interactions the structure of the folding oligonucleotide expands spaciously to an open circle structure. In the open circle structure said first and third sequences of nucleic acids face and complement the sequence of the target and said second sequence of nucleic acids is turned upwards facing away from the target. This second sequence of nucleic acids may either present a sequence identical with the wild-type sequence thereby rectifying the mutation, or it may comprise instead of or in addition, a heterologous sequence which is different from the sequence of the wildtype pre-mRNA or RNA target molecule thereby introducing to the endogenous molecule a heterologous element or motif, e.g., a sequence motif that serves as a recognition site for an RNA binding protein. The second sequence of nucleic acids may generate tertiary structures, based on the sequence of the nucleotides.

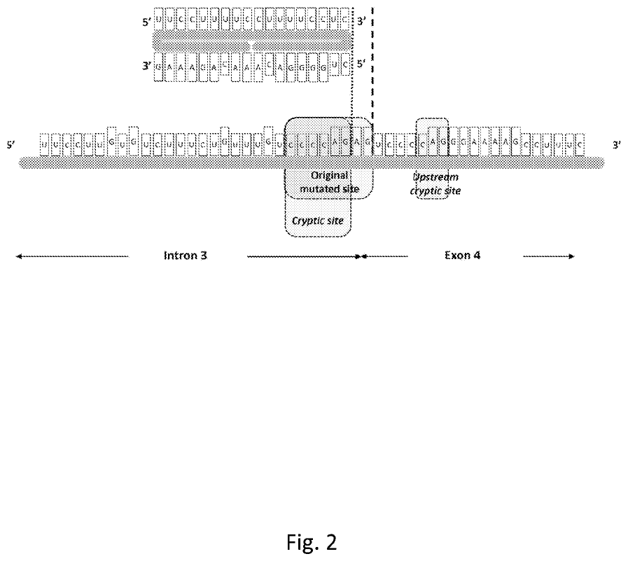

One embodiment of the folding oligonucleotide of the invention is illustrated in FIG. 2, showing the open circle folding oligonucleotide in a compressed form.

In accordance with this embodiment of the invention, the folding oligonucleotide comprises a first sequence of nucleic acids that hybridizes with a sequence downstream the cryptic area, and is a part of the PPT;

A second sequence of nucleic acids being directed upwards and having a sequence which mimics and is in the same 5′->3′ direction as the PPT sequence of the wildtype pre-mRNA; and

A third sequence of nucleic acids that hybridizes with and masks the upstream cryptic area (see in FIG. 1 an annotation of the upstream cryptic area).

In an embodiment, at least one of the sequences of nucleic acids that is complementary to the target pre-mRNA or mRNA is of a length that determines high specificity and strong hybridization capability (a non-limiting example is a sequence of about 15 nucleotides) and the second sequence of nucleic acids that is complementary to the target pre-mRNA or mRNA may be either a short sequence of less specificity or it may be of a length that also determines high specificity and strong hybridization capability.

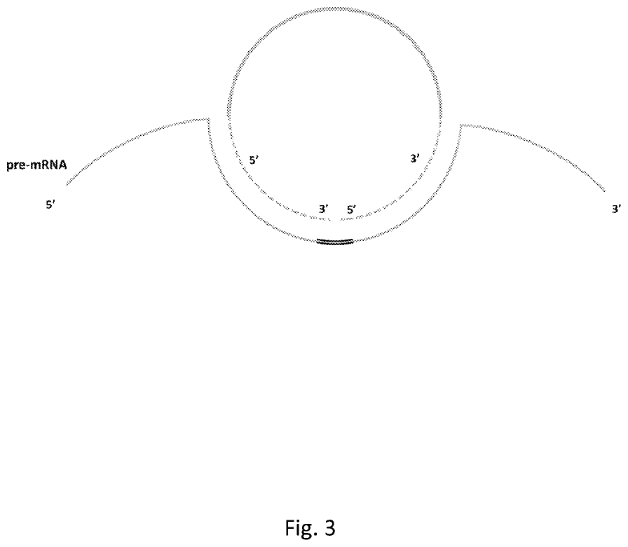

In one embodiment, the length of the second sequence of nucleic acids which is exposed upwards is identical to the accumulated lengths of the flaps and the heterologous sequence is identical with the wildtype sequence thereby rectifying the mutation. This embodiment is illustrated in FIG. 3 showing as an example the rectification of a point mutation.

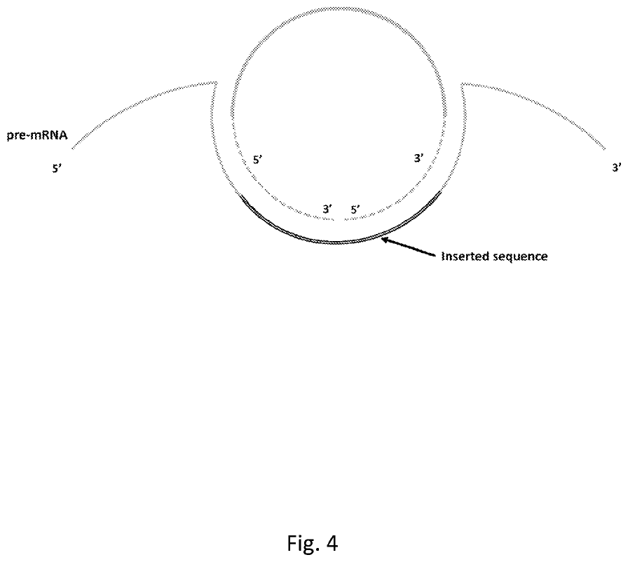

In another embodiment, the folding oligonucleotide of the invention is used to rectify an insertion mutation. In such case, the length of the second sequence of nucleic acids which is exposed upwards is shorter than the accumulated lengths of the flaps and the heterologous sequence is identical with the wildtype sequence thereby rectifying the mutation. This embodiment is illustrated in FIG. 4.

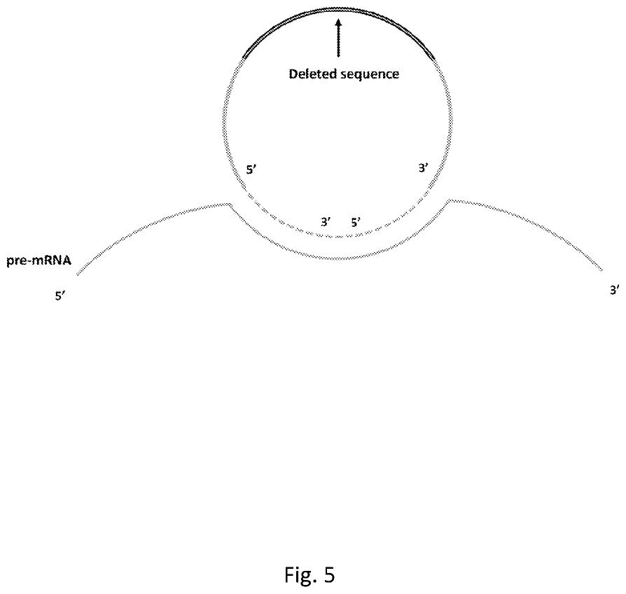

In another embodiment, the folding oligonucleotide of the invention is used to rectify a deletion mutation. In such case, the length of the second sequence of nucleic acids which is exposed upwards is longer than the accumulated lengths of the flaps and the heterologous sequence is identical with the wildtype sequence thereby rectifying the mutation. This embodiment is illustrated in FIG. 5.

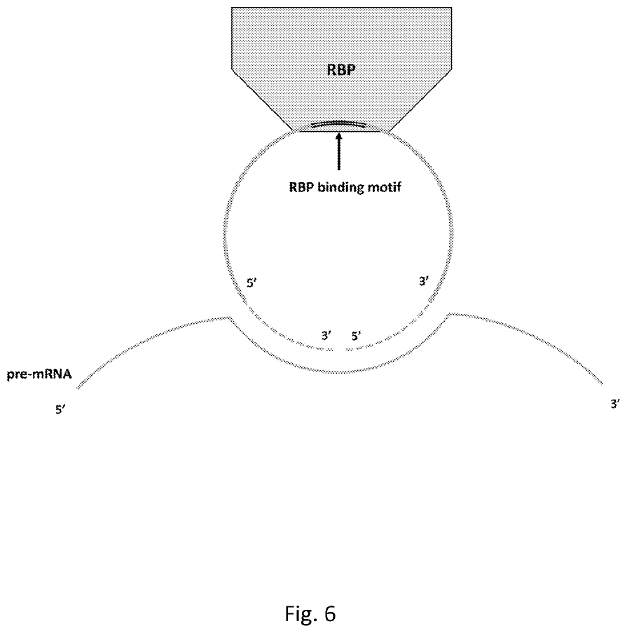

In another embodiment, the folding oligonucleotide of the invention is used for introducing a heterologous motif to the target mRNA or pre-mRNA molecule together with or instead of rectifying a mutation. In such case, the length of the second sequence of nucleic acids which is exposed upwards may be longer or shorter than the accumulated lengths of the flaps and the heterologous sequence comprises a sequence motif that serves as a recognition site for an RNA binding element. e.g., an RNA binding protein that can serve as a recognition site for attracting various enzymes or ribozymes which may affect the translation procedure. This embodiment is illustrated in FIG. 6.

The open circle folding oligonucleotide refers to a circular structure that is not closed. However, in one embodiment, the folding oligonucleotide is chemically closed, to generate a completely circular molecule.

In one embodiment, the folding oligonucleotide is from about 40 to about 200 bases long.

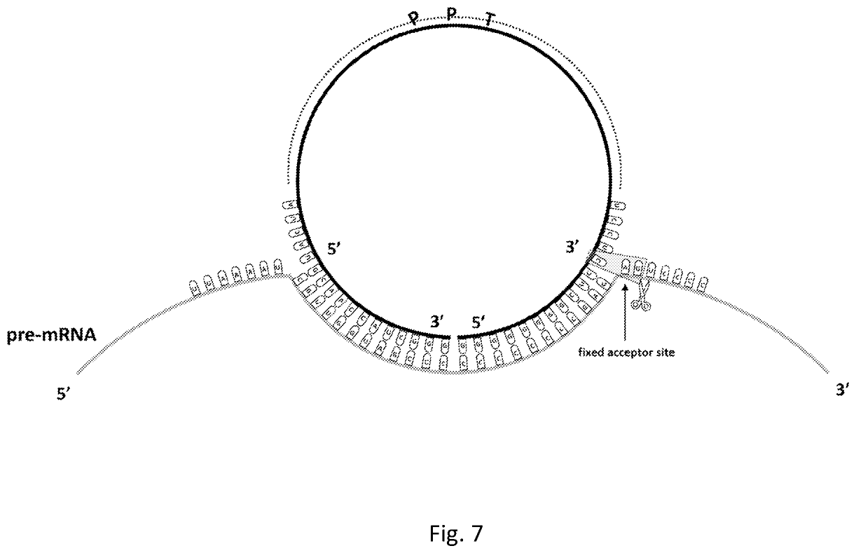

FIGS. 7-9 provide schematic illustrations of various embodiments of the open circle folding oligonucleotide of the invention. In these specific exemplary embodiments, the folding oligonucleotide rectifies a C to G mutation by replacing the G back to the wild-type C. The figures show schematically the spatial circular structure formed by the folding oligonucleotide

FIG. 7 is a schematic illustration of one embodiment of the invention showing an open circle folding oligonucleotide in which the G mutation is masked with a hybridizing C.

C is added to the end of the exposed part of the Ocirc folding oligonucleotide, preceding the AG of the original acceptor site.

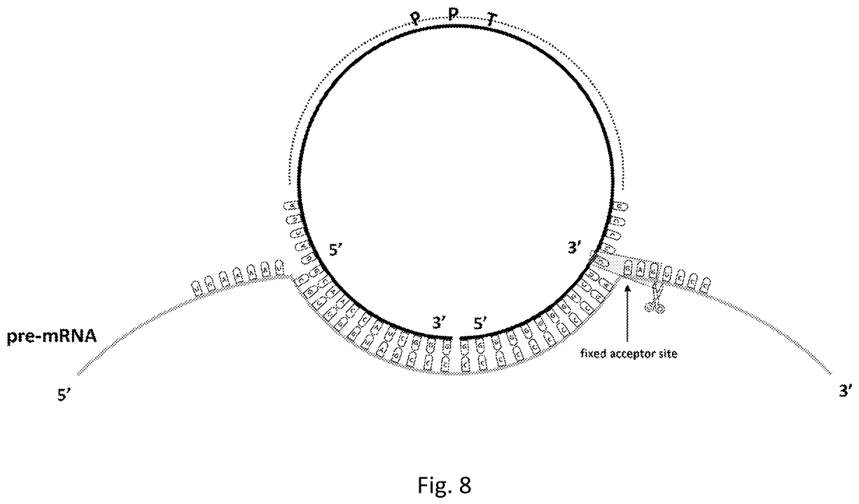

FIG. 8 is a schematic illustration of another embodiment of the invention showing an open circle folding oligonucleotide in which the G mutation is kept, but an inverted C nucleotide is placed at the end of the exposed part of the Ocirc folding oligonucleotide, aligned with the mutated G.

Inverted base oligonucleotides are oligonucleotides with 5′-5′ or 3′-3′ linkages or a combination of these in the same oligo.

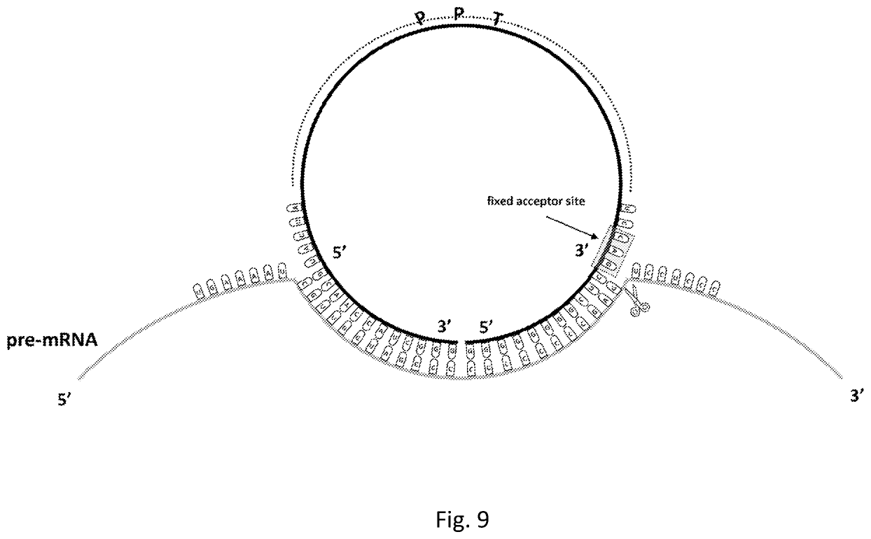

FIG. 9 is a schematic illustration of another embodiment of the invention showing an open circle folding oligonucleotide in which the mutated GAG acceptor site sequence is masked.

A correct CAG sequence is placed at the end of the exposed part of the Ocirc folding oligonucleotide.

Example 1 below shows the sequences of three representative folding oligonucleotide molecules (1, 2, and 3) corresponding respectively to the folding oligonucleotide schematically represented in FIGS. 7-9.

An alternative splicing acceptor site may be designed using dedicated tools. e.g., NetGene2, e.g., as shown in Example 1 below presenting NetGene2 simulation results estimating the confidence of potential splicing acceptor sites. In one embodiment, the alternative splicing acceptor site is represented by Seq ID NO: 31.

The sequences are planned to achieve optimal results, while attempting to minimize “stacking” of the folding oligonucleotides to one another, or unwanted hybridization of parts in a folding oligonucleotide molecule.

Various solutions may be employed to reduce the stacking, all of which involve the introduction of nucleic acid alterations into the sequence to avoid further binding of the folding oligonucleotides to one another. For example, one solution may comprise introducing minute changes, i.e., one or more nucleic acid substitutions, in the selected PPT sequence thus presenting a PPT which is slightly different from the native, wildtype sequence, yet maintaining the features of a strong PPT. Another solution, relevant to cases where the mutation is in the exon, would be to introduce nucleic acid substitutions, which would alter the nucleic acid sequence buy yet, maintain the codon reading. This solution is based on the codon redundancy, namely that different sets of codons can encode the same amino acid. Thereby, a correct amino acid sequence is maintained although the nucleic acid sequence is altered.

In another embodiment the open circle folding oligonucleotide may further comprise binding sites for RNA binding proteins. In an aspect of the invention the folding oligonucleotide may act via a trans-splicing mechanism.

The Ocirc molecules of the invention may comprise one or more modified nucleotides to increase the molecule's stability. Modified nucleotides include, but are not limited to, 2′-O-methyl modified nucleotides, LNA (Locked Nucleic Acids) modified nucleotides or 2′MOE (2′-O-methoxy ethyl/phosphorothioate) modified nucleotides. One, two, three, four, five or six nucleotides can be incorporated at either end of the Ocirc arms. Furthermore, the entire arms can be composed of modified nucleotides and selected nucleotides from the rest of the Ocirc can also be modified nucleotides.

### Folding Oligonucleotide for Trans-Splicing

In known methods of trans-splicing, to achieve the trans-splicing event, the antisense oligomer (also referred to as the antisense oligonucleotide) (ASO) must include the entire exon which is typically several hundreds of nucleotides long. The reason for that is that the trans-splicing event relies on the natural splicing cues that are located at the intron-exon junctions.

In contrast, the folding oligonucleotide in accordance with the present invention may be shorter and does not necessarily comprise the full exon sequence. In certain embodiments, it may be between about 100-200 nucleotides long.

The trans-splicing in accordance with the invention will not occur in the original authentic splice sites but will rather employ “pseudo” acceptor and donor sequences that are present within the relevant exon.

Accordingly, the folding oligonucleotide of the invention comprises an alternative splice acceptor site, followed by a sequence identical with the target exon (referred to as “an artificial exon” or “synthetic exon”). If the mutation is in the intron, the folding oligonucleotide of the invention will mask the mutated area, generate a new splice point, and will further comprise a sequence identical with the “disabled” sequence of the wild-type target exon.

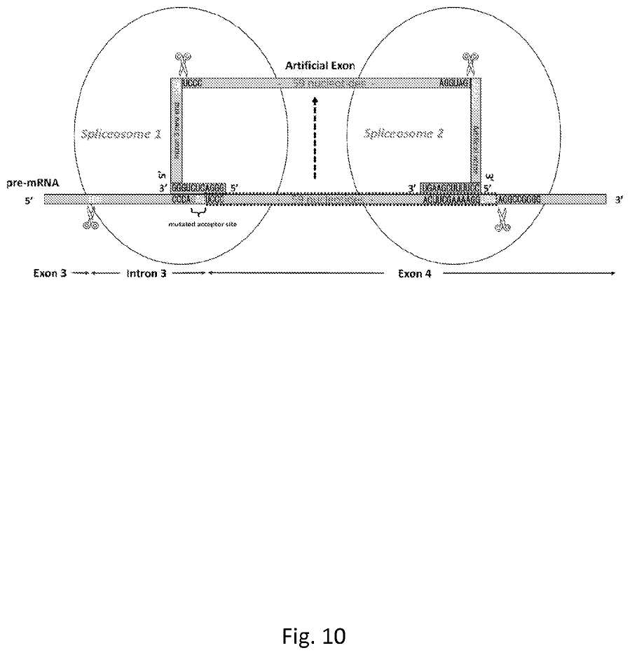

FIG. 10 describes a schematic illustration of an embodiment of the invention. Accordingly, if the mutation is in the intron (for example in the acceptor site), the trans-splicing event will replace the mutated sequence with the artificial exon having the wild-type sequence. According to this embodiment, folding oligonucleotide comprises: a first sequence that hybridizes with the pre-mRNA and which optionally masks potential cryptic sites, a portion of an artificial intron that includes an acceptor site, an artificial exon that replaces a portion of the original exon, a portion of an artificial intron whose acceptor site is derived from the original exon, and another sequence that hybridizes with the pre-mRNA.

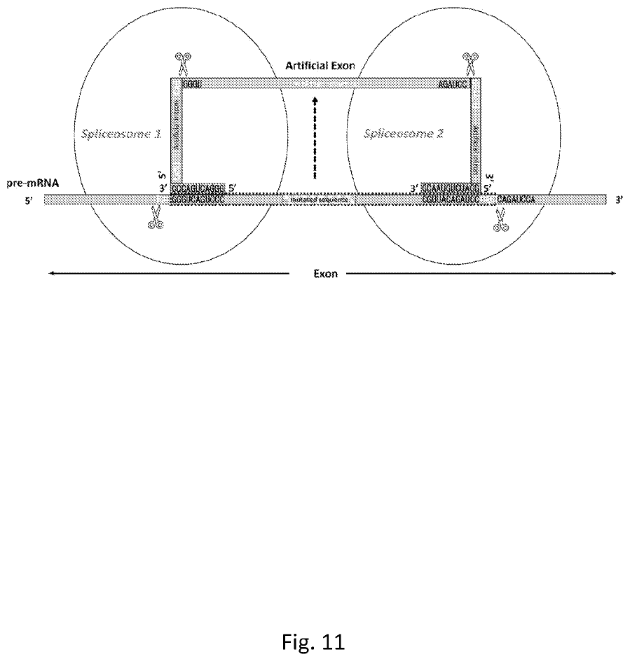

If the mutation is in the exon, the trans-splicing event will replace the mutated sequence with the artificial exon having the wild-type sequence. According to this embodiment, both the donor site and the acceptor site are derived from the nucleic acid sequence of the original, mutated exon. A schematic illustration of this embodiment is shown in FIG. 11.

To illustrate the trans-splicing event in accordance with the invention, FIG. 1 provides a schematic illustration of an exemplary intronic mutation showing a C to G mutation in a pre-mRNA transcript. This mutation causes the activation of a cryptic splice site, two nucleotides upstream of the correct, authentic splice site. The activation of the cryptic splice site causes a frame shift resulting in a mutated mRNA transcript.

As demonstrated in FIG. 10, the folding oligonucleotide of the invention comprises an alternative splice acceptor site instead of the mutated acceptor site (e.g., the GAG cryptic site as shown in FIG. 1), followed by an artificial exon that is identical with the relevant part of the target exon.

The folding oligonucleotide further comprises an artificial intron sequence, ending with a polypyrimidine tract (PPT) next to a YAG acceptor site (a conserved 3′ splice site essential for the pre-mRNA splicing) that is part of the original exon sequence.

The invention therefore provides a folding oligonucleotide comprising from 5′ to 3′.

A first complementary sequence of between about 10 and 15 bases (e g., ˜12 bases) long that complements and is capable of hybridizing with the mutation area, wherein the mutation area comprises the mutation site, the downstream cryptic site, and the upstream cryptic site;

A trans-splicing alternative acceptor site, comprising a preceding strong PPT;

A sequence which is identical with the original exon sequence between the end of the first complementary sequence at the 5′ terminus of the folding oligonucleotide, and the 3 nucleotides (the YAG acceptor site) following the second complementary sequence (at the 3′ terminus of the folding oligonucleotide);

An artificial intron that includes a donor site, branch point and PPT, ending close to the YAG sequence that is part of the original exon;

A second complementary sequence of between about 10 and 15 bases (e.g., ˜12 bases) long that complements and is capable of hybridizing with the sequence that precedes the YAG acceptor site.

As used herein the term “strong PPT” refers to a polypyrimidine tract (PPT) that is capable of strongly attracting (e.g., being competitively advantageous in attracting) the spliceosome to perform splicing at the splice site adjacent to the PPT. The PPT is an important cis-acting sequence element directing intron removal in pre-mRNA splicing. There appears to be great flexibility in the specific sequence of a PPT, with diverse levels of functional competitive efficiency in directing the spliceosome to the splice-point. There are known methods for preparing strong PPT, for example pyrimidine tracts containing II continuous uridines were found to be very strong pyrimidine tracts (Coolidge et al., (1997) Nucleic Acid Res. 25(4): 888-896).

In an embodiment, at least one of the sequences of nucleic acids that is complementary to the target pre-mRNA or mRNA is of a length that determines high specificity and strong hybridization capability (a non-limiting example is a sequence of about 15 nucleotides) and a second sequence of nucleic acids that is complementary to the target pre-mRNA or mRNA that may be either a short sequence of less specificity or it may be of a length that also determines high specificity and strong hybridization capability. This embodiment is schematically illustrated in FIG. 12, which demonstrates the rectification of a mutation in the acceptor site.

In general, the GURAGU donor site can be also located in an exon and continued in the first artificial intron inside the folding oligonucleotide. Since there is much higher chance to find GU or GUR sequence in an exon than the full donor sequence, the required sequence may split between the exon and the folding oligonucleotide. The same applies for the acceptor site (continuing the second artificial intron), which its full sequence is YNCAG. (R: A or G, Y: C or T, N: any nucleotide). The folding oligonucleotide harbors the exon sequence that will replace the original one encoded by the endogenous gene, a 5′ and/or a 3′ splice site whose strength must be equivalent to, or even stronger than, the one carried by the pre-mRNA.

The folding oligonucleotide may be introduced in the cell by any method known in the art, non-limiting examples include transfecting a plasmid carrying the gene that expresses the folding oligonucleotide, using a recombinant viral vector (e.g., adeno associated virus (AAV)) that will express the folding oligonucleotide, lipid encapsulation and the like. Special delivery vehicles would be selected for introducing the folding oligonucleotide into the brain, in cases that the correction of the mRNA or pre-mRNA is required in the brain. Such vehicles would be chosen based on their ability to cross the blood brain barrier (BBB) and be injected via the spinal cord.

The recombinant AAV (rAAV) vectors in accordance with the invention are typically composed of, at a minimum, a transgene (namely, the folding oligonucleotide of the invention) operatively linked to regulatory sequences which permit its expression in a cell of a target tissue, and 5′ and 3′ AAV inverted terminal repeats.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. Disclosed and described, it is to be understood that this invention is not limited to the specific examples, methods steps, and compositions disclosed herein as such methods steps and compositions may vary somewhat. It is also to be understood that the terminology used herein is used for the purpose of describing specific embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### Example 1: Simulation of Representative Folding Oligonucleotides

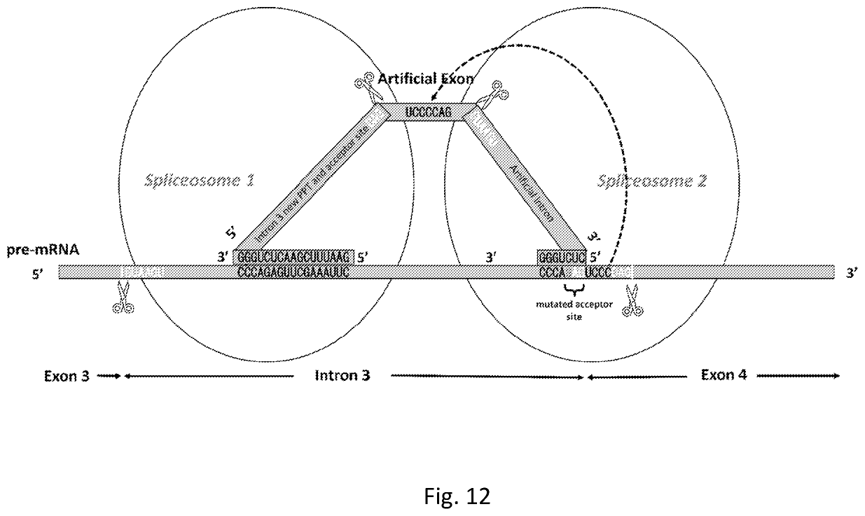

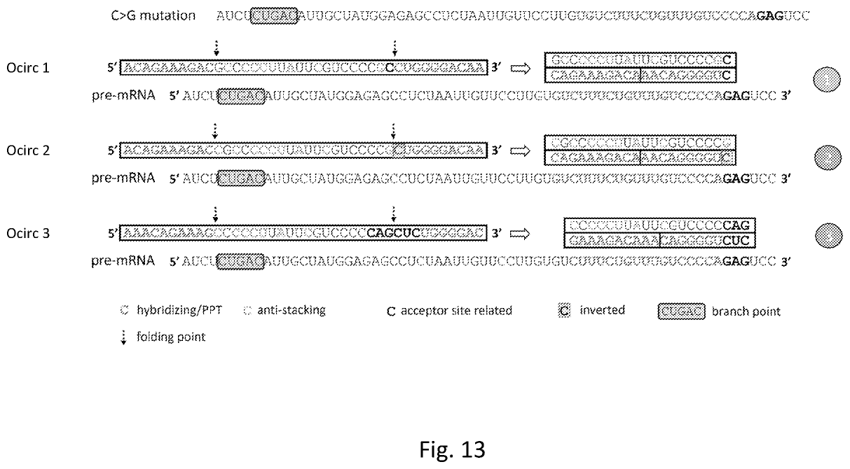

Representative, exemplary, folding oligonucleotide molecules designated Ocirc 1, Ocirc 2, and Ocirc 3 (corresponding respectively to SEQ ID Nos 12-14) were constructed as shown in FIG. 13 (options 1, 2, and 3) These oligonucleotide molecules correspond respectively to the folding oligonucleotide schematically represented in FIGS. 7-9.

The “arms” of these folding oligonucleotide molecules were designed to pair with the sequence bridging the end of the 3^{rd} intron and the beginning of the 4^{th} exon of MECP2.

As indicated in FIGS. 7-9 the folding oligonucleotides comprise a PPT portion.

According to these options, part of the original PPT sequence of intron 4 is replaced with a new sequence which is part of the Ocirc molecule.

To design an optimal PPT portion, a splicing simulation was performed using Netgene2, a software tool which predicts splicing points based on given sequences of introns and exons.

To search for sequences with a potential splice site, the following sequence (designated SEQ ID NO: 27) originating from Homo sapiens chromosome X, GRCh38.p13, NC_000023.11: c154031955-154030936 was used for the simulation:

GGAACTTGCAGAGCTAGGGGTTCAGAGGGGTGAAGAAGCATGTTTCAGTT
CTGCCTTTTAAATGATCCCAAAAAGGTTAGCAGTTTTCAAATGACATTTG
CAGACAGCCTCATTTAATTCCATGAGAAGGGTGAGCAAAGGATTATCTTG
TTGAAACTGATTCCTGGAGAGACTGAGCACCGTACCTGAGTTCAAACTTG
GGAATGTTCTAGATGGTGACTCAGGCCCAGGCACCAACCAGCAGAATGGG
CCTCAGCCTGACAACCCTTCTGTACCAGGCCTGACTCTTTGGTTGCTGAA
CTTTGGAGAGGCCTGGGGGGGTCAGCGGCAGGCAGACGAGTGAGTGGCTT
TGGTGACAGGTCCTCAGGGGCAGCCAGGCAGTGTGACTCTCGTTCAATAG
TAACGTTTGTCAGAGCGTTGTCACCACCATCCGCTCTGCCCTATCTCTGA
CATTGCTATGGAGAGCCTCTAATTGTTCCTTGTGCCCCCTTATTCGTCCC
CGCAGTCCCCAGGGAAAAGCCTTTCGCTCTAAAGTGGAGTTGATTGCGTA
CTTCGAAAAGGTAGGCGACACATCCCTGGACCCTAATGATTTTGACTTCA
CGGTAACTGGGAGAGGGAGCCCCTCCCGGCGAGAGCAGAAACCACCTAAG
AAGCCCAAATCTCCCAAAGCTCCAGGAACTGGCAGAGGCCGGGGACGCCC
CAAAGGGAGCGGCACCACGAGACCCAAGGCGGCCACGTCAGAGGGTGTGC
AGGTGAAAAGGGTCCTGGAGAAAAGTCCTGGGAAGCTCCTTGTCAAGATG
CCTTTTCAAACTTCGCCAGGGGGCAAGGCTGAGGGGGGTGGGGCCACCAC
ATCCACCCAGGTCATGGTGATCAAACGCCCCGGCAGGAAGCGAAAAGCTG
AGGCCGACCCTCAGGCCATTCCCAAGAAACGGGGCCGAAAGCCGGGGAGT
GTGGTGGCAGCCGCTGCCGCCGAGGCCAAAAAGAAAGCCGTGAAGGAGTC
TTCTATCCGATCTGTGCAGG

The following sequences (which are fragments of the above basic sequence) were selected by the simulation tool as potential splice sites:

(SEQ ID NO: 28)
AATGTTCTAG\hat{ }ATGGTGACTC
(SEQ ID NO: 29)
GGTGACTCAG\hat{ }GCCCAGGCAC
(SEQ ID NO: 30)
TCAGGCCCAG\hat{ }GCACCAACCA
(SEQ ID NO: 31)
GTCCCCGCAG\hat{ }TCCCCAGGGA
(SEQ ID NO: 32)
CAGTCCCCAG\hat{ }GGAAAAGCCT
(SEQ ID NO: 33)
CAGGGAAAAG\hat{ }CCTTTCGCTC
(SEQ ID NO: 34)
CGCTCTAAAG\hat{ }TGGAGTTGAT
(SEQ ID NO: 35)
TAAAGTGGAG\hat{ }TTGATTGCGT
(SEQ ID NO: 36)
ATCCACCCAG\hat{ }GTCATGGTGA
(SEQ ID NO: 37)
GCCCCGGCAG\hat{ }GAAGCGAAAA
(SEQ ID NO: 38)
CGGCAGGAAG\hat{ }CGAAAAGCTG
(SEQ ID NO: 39)
AAGCGAAAAG\hat{ }CTGAGGCCGA

The ∧ symbol represents the intersection between the intron (on the left-hand side) and the exon (on the right-hand side).

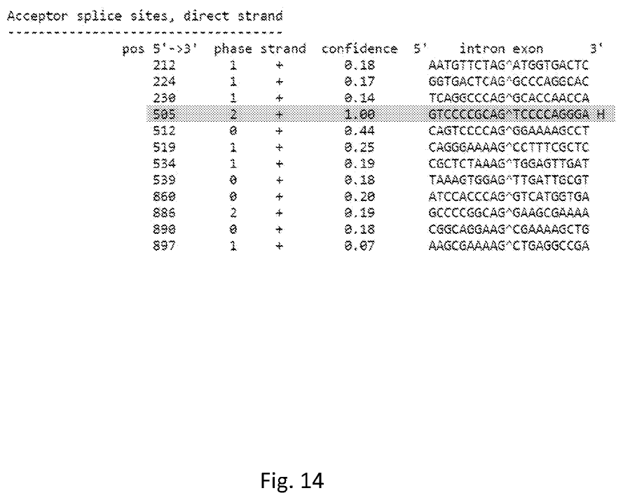

Results of the analysis are presented in FIG. 14. The confidence score is a numerical value that typically ranges from 0 to 1, where higher values indicate a higher level of confidence in the prediction. Namely, a higher confidence score suggests a higher likelihood that the predicted splice site is accurate. The “phase” may have one of three values: 0, 1 or 2.

A phase 0 splice site indicates that the predicted splice site corresponds to the canonical phase for splicing. In other words, the intron-exon boundary aligns correctly with the reading frame, ensuring that the protein-coding sequence is not disrupted during translation. A phase 0 splice site is the most common and preferred phase in many genes.

A phase 1 splice site suggests that the intron-exon boundary is shifted by one nucleotide compared to the canonical phase. This shift can result in a slight disruption of the reading frame, potentially leading to a different amino acid sequence in the protein product.

A phase 2 splice site indicates that the intron-exon boundary is shifted by two nucleotides relative to the canonical phase. This results in a more significant disruption of the reading frame, potentially leading to a different amino acid sequence and often introducing a premature stop codon, which can affect protein functionality.

Understanding the phase of a predicted splice site is crucial for accurate gene annotation and predicting the functional consequences of splice site variations. Researchers and biologists can use this information to assess how a given mutation or alternative splice site might affect the final protein product and its function.

Based on the confidence value, the highlighted sequence in FIG. 14 (SEQ ID NO: 31) was selected as having the highest likelihood for being a splice site (indicated by the letter H).

### Example 2: Preparation of the Template Plasmids which Serve as Targets for the Ocirc Oligonucleotides

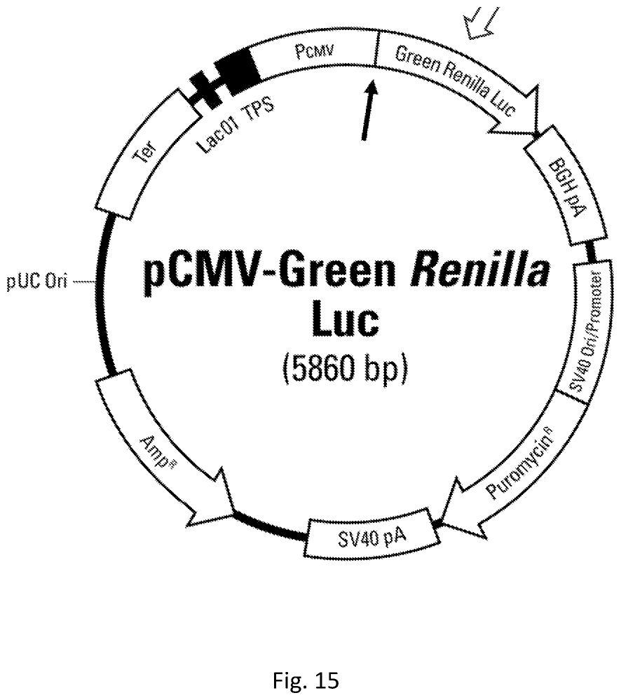

All plasmids were constructed based on the same original plasmid: pCMV-Green Renilla Luc. The plasmid was purchased from ThermoFisher scientific, Catalog number: 16153. A map of the plasmid is shown in FIG. 15.

The following features are present in the plasmid based on its nucleotide sequence referred to herein as SEQ ID NO: 1:
Cytomegalovirus (CMV) promoter: 8-635
Green Renilla luciferase gene: 646-1581
BGH poly(A) signal: 1590-1715
SV40 origin/promoter: 1716-2280
Puromycin resistant gene: 2281-2880
SV40 poly(A) signal: 3042-3075
beta-lactamase (Amp^{R}) gene: 3184-4044
pUC replication origin (pUC Ori): 4223-5027
Transcriptional terminator (Ter): 5028-5635
Lac operator 1 (Lac 01): 5636-5656
Transcriptional pause site (TPS): 5789-5860

The sequence of the plasmid (designated SEQ ID NO:1) is as follows:

ACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTG
GCACCAAAATCAACGGGACTTTCCAAAATGTCGTAACAACTCCGCCCCATTGACGCAAA
TGGGCGGTAGGCGTGTACGGTGGGAGGTCTATATAAGCAGAGCTCTCTGGCTAACTAGA
GAACCCACTGCTTACTGGCTTATCGAAATTAATACGACTCACTATAGGGGATCCGCCAC
CATGGCCAGCAAGGTGTACGACCCCGAGCAGCGGAAGCGGATGATCACCGGCCCTCAGT
GGTGGGCTCGGTGCAAGCAGATGAACGTGCTGGACAGCTTCATCAACTACTACGACAGC
GAGAAGCACGCCGAGAACGCCGTGATCTTCCTGCACGGCAACGCCACCAGCAGCTACCT
GTGGCGGCACGTGGTGCCCCACATCGAGCCTGTGGCCAGATGCATCATCCCCGACCTGA
TCGGCATGGGCAAGAGCGGCAAGTCCGGCAACGGCAGCTACCGGCTGCTGGACCACTAC
AAGTACCTGACCGCTTGGTTTGAGCTGCTGAACCTGCCCAAGAAGATCATCTTCGTCGG
CCACGACTGGGGCAGCGCCCTGGCCTTTCACTACGCCTACGAGCACCAGGACCGGATCA
AGGCCATCGTGCACATGGAAAGCGTGGTGGACGTGATCGAGAGCTGGATGGGCTGGCCC
GACATCGAGGAAGAACTGGCCCTGATCAAGAGCGAAGAGGGCGAGAAGATGGTGCTGGA
AAACAACTTCTTCGTGGAAACCCTGCTGCCCAGCAAGATCATGCGGAAGCTGGAACCCG
AAGAGTTCGCCGCCTACCTGGAACCCTTCAAAGAAAAGGGCGAAGTGCGGAGGCCCACC
CTGAGCTGGCCCAGAGAGATCCCCCTGGTCAAGGGCGGCAAGCCCGACGTGGTGCAGAT
CGTGCGGAACTACAACGCCTACCTGCGGGCCAGCGACGACCTGCCTAAGCTGTTCATCG
AGAGCGACCCCGGCTTCTTCAGCAACGCCATCGTGGAAGGCGCCAAGAAGTTCCCCAAC
ACCGAGTTCGTGAAAGTGAAGGGCCTGCACTTCCTCCAGGAAGATGCCCCCGACGAGAT
GGGCAAGTACATCAAGAGCTTCGTGGAACGGGTGCTGAAGAACGAGCAGTGAGCGGCCG
CAAAATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGGA
AATTGCATCACAACACTCAACCCTATCTCGGTCTATTCTTTTGATTTATAAGGGATTTT
GCCGATTTCGGCCTATTGGTTAAAAAATGAGCTGATTTAACAAAAATTTAACGCGAATT
AATTCTGTGGAATGTGTGTCAGTTAGGGTGTGGAAAGTCCCCAGGCTCCCCAGCAGGCA
GAAGTATGCAAAGCATGCATCTCAATTAGTCAGCAACCAGGTGTGGAAAGTCCCCAGGC
TCCCCAGCAGGCAGAAGTATGCAAAGCATGCATCTCAATTAGTCAGCAACCATAGTCCC
GCCCCTAACTCCGCCCATCCCGCCCCTAACTCCGCCCAGTTCCGCCCATTCTCCGCCCC
ATGGCTGACTAATTTTTTTTATTTATGCAGAGGCCGAGGCCGCCTCTGCCTCTGAGCTA
TTCCAGAAGTAGTGAGGAGGCTTTTTTGGAGGCCTAGGCTTTTGCAAAAAGCTCCCGGG
AGCTTGTATATCCATTTTCGGATCTGATCAGCACGTGTTGACAATTAATCATCGGCATA
GTATATCGGCATAGTATAATACGACAAGGTGAGGAACTAAACCATGACCGAGTACAAGC
CCACGGTGCGCCTCGCCACCCGCGACGACGTCCCCAGGGCCGTACGCACCCTCGCCGCC
GCGTTCGCCGACTACCCCGCCACGCGCCACACCGTCGATCCGGACCGCCACATCGAGCG
GGTCACCGAGCTGCAAGAACTCTTCCTCACGCGCGTCGGGCTCGACATCGGCAAGGTGT
GGGTCGCGGACGACGGCGCCGCGGTGGCGGTCTGGACCACGCCGGAGAGCGTCGAAGCG
GGGGCGGTGTTCGCCGAGATCGGCCCGCGCATGGCCGAGTTGAGCGGTTCCCGGCTGGC
CGCGCAGCAACAGATGGAAGGCCTCCTGGCGCCGCACCGGCCCAAGGAGCCCGCGTGGT
TCCTGGCCACCGTCGGCGTCTCGCCCGACCACCAGGGCAAGGGTCTGGGCAGCGCCGTC
GTGCTCCCCGGAGTGGAGGCGGCCGAGCGCGCCGGGGTGCCCGCCTTCCTGGAGACATC
CGCGCCCCGCAACCTCCCCTTCTACGAGCGGCTCGGCTTCACCGTCACCGCCGACGTCG
AGGTGCCCGAAGGACCGCGCACCTGGTGCATGACCCGCAAGCCCGGTGCCTGACACGTG
CTACGAGATTTCGATTCCACCGCCGCCTTCTATGAAAGGTTGGGCTTCGGAATCGTTTT
CCGGGACGCCGGCTGGATGATCCTCCAGCGCGGGGATCTCATGCTGGAGTTCTTCGCCC
ACCCCAACTTGTTTATTGCAGCTTATAATGGTTACAAATAAAGCAATAGCATCACAAAT
TTCACAAATAAAGCATTTTTTTCACTGCATTCTAGTTGTGGTTTGTCCAAACTCATCAA
TGTATCTTATCATGTCTGTATACCGTCGACCTCTAGCTAGAGCTTGGCGTAATCATGGT
CATTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTG
GCCCCAGCGCTGCGATGATACCGCGAGAACCACGCTCACCGGCTCCGGATTTATCAGCA
ATAAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTT
TGCGCAACGTTGTTGCCATCGCTACAGGCATCGTGGTGTCACGCTCGTCGTTTGGTATG
GCTTCATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTGTATGCG
GCGACCGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAA
AGGGAATAAGGGCGACACGGAAATGTTGAATACTCATATTCTTCCTTTTTCAATATTAT
TGAAGCATTTATCAGGGTTATTGTCTCATGAGCGGATACATATTTGAATGTATTTAGAA
AAATAAACAAATAGGGGTCAGTGTTACAACCAATTAACCAATTCTGAACATTATCGCGA
GCCCATTTATACCTGAATATGGCTCATAACACCCCTTGCTCATGACCAAAATCCCTTAA
CGTGAGTTACGCGCGCGTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGTAGTTAGCC
CACCACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACGATAGT
TACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTC
ACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGA
AGCGGAAGGCGAGAGTAGGGAACTGCCAGGCATCAAACTAAGCAGAAGGCCCCTGACGG
ATGGCCTTTTTGCGTTTCTACAAACTCTTTCTGTGTTGTAAAACGACGGCCAGTCTTAA
GCTCGGGCCCCCTGGGCGGTTCTGATAACGAGTAATCGTTAATCCGCAAATAACGTAAA
AACCCGCTTCGGCGGGTTTTTTTATGGGGGGAGTTTAGGGAAAGAGCATTTGTCAGAAT
ATTTAAGGGCGCCTGTCACTTTGCTTGATATATGAGAATTATTTAACCTTATAAATGAG
AAAAAAGCAACGCACTTTAAATAAGATACGTTGCTTTTTCGATTGATGAACACCTATAA
TTAAACTATTCATCTATTATTTATGATTTTTTGTATATACAATATTTCTAGTTTGTTAA
AGAGAATTAAGAAAATAAATCTCGAAAATAATAAAGGGAAAATCAGTTTTTGATATCAA
AATTATACATGTCAACGATAATACAAAATATAATACAAACTATAAGATGTTATCAGTAT
TTATTATCATTTAGAATAAATTTTGTGTCGCCCTTAATTGTGAGCGGATAACAATTACG
AGCTTCATGCACAGTGGCGTTGACATTGATTATTGACTAGCATGTTCTTTCCTGCGTTA
TCCCCTGATTCTGTGGATAACCGTATTACCGCCATGCATTAGTTATTAATAACATACGC
TCTCCATCAAAACAAAACGAAACAAAACAAACTAGCAAAATAGGCTGTCCCCAGTGCAA
GTGCAGGTGCCAGAACATTTCTCT

Several derivative plasmids were produced from the basic plasmid (by GeneScript, Singapore).

All derivative plasmids had an insertion of the following region of human beta globin 5′UTR (hBB) (designated SEQ ID NO: 2) between the CMV promoter and the Green Renilla Luc gene. As can be seen in FIG. 15, the black arrow marks the insertion point.

SEQ ID NO: 2:
5′ACATTTGCTTCTGACACAACTGTGTTCACTAGCAACCTCAAACAGACA
CC-3′

In addition, an intron sequence was inserted into the plasmid at the Green Renilla Luciferase (Luc) gene as will be specified below.

### Plasmid DCMV-RLuc-Int WT

An intron was inserted into the following location of the Green Renilla Luc gene:

GATCTGATAG-“intron”-GTATGGGCAA

This insertion point was selected since it contains sequences characteristic of exon ends (AG) and exon beginning (GT). These are marked as bold, underlined in the above sequence.

The following “intron” sequence (designated SEQ ID NO: 3) was inserted at the location indicated above:

GTTGGTATCAAGGTTACAAGACAGGTTTAAGGAGACCAATAGAAACTGGG
CATGTGGAGACAGAGAAGACTCTTGGGTTTCTGAGAGAGGCCTGGGGGGG
TCAGCGGCAGGCAGACGAGTGAGTGGCTTTGGTGACAGGTCCTCAGGGGC
AGCCAGGCAGTGTGACTCTCGTTCAATAGTAACGTTTGTCAGAGCGTTGT
CACCACCATCCGCTCTGCCCTATCTCTGACATTGCTATGGAGAGCCTCTA
ATTGTTCCTTGTGTCTTTCTGTTTGTCCCCACAG

The 1^{st} part of the inserted intron (underlined) is composed of 84 nucleotides from the 5′ end of the 1^{st} intron of the human beta globin gene (sequence taken from the human genome presented in the UCSC genome web site).

The 2^{nd} part of the intron (bold) is composed of 200 nucleotides from the 3′ region of the 3^{rd} intron of the human MECP2 gene.

After the insertion the derivative plasmid termed “Plasmid pCMV-RLuc-Int_WT” had the following sequence (designated SEQ ID NO: 4):

ACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTG
GCACCAAAATCAACGTCAGAGCGTTGTCACCACCATCCGCTCTGCCCTATCTCGGGACT
TTCCAAAATGTCGTAACAACTCCGCCCCATTGACGCAAATGGGGGGTAGGCGTGTACGG
TGGGAGGTCTATATAAGCAGAGCTCTCTGGCTAACTAGAGAACCCACTGCTTACTGGCT
TATCGAAATTAATACGACTCACTATAGGGGATCCACATTTGCTTCTGACACAACTGTGT
TCACTAGCAACCTCAAACAGACACCATGGCCAGCAAGGTGTACGACCCCGAGCAGCGGA
AGCGGATGATCACCGGCCCTCAGTGGTGGGCTCGGTGCAAGCAGATGAACGTGCTGGAC
AGCTTCATCAACTACTACGACAGCGAGAAGCACGCCGAGAACGCCGTGATCTTCCTGCA
CGGCAACGCCACCAGCAGCTACCTGTGGCGGCACGTGGTGCCCCACATCGAGCCTGTGG
CCAGATGCATCATCCCAGATCTGATAGGTTGGTATCAAGGTTACAAGACAGGTTTAAGG
AGACCAATAGAAACTGGGCATGTGGAGACAGAGAAGACTCTTGGGTTTCTGAGAGAGGC
CTGGGGGGGTCAGCGGCAGGCAGACGAGTGAGTGGCTTTGGTGACAGGTCCTCAGGGGC
AGCCAGGCAGTGTGACTCTCGTTCAATAGTAACGTTTGTCAGAGCGTTGTCACCACCAT
CCGCTCTGCCCTATCTCTGACATTGCTATGGAGAGCCTCTAATTGTTCCTTGTGTCTTT
CTGTTTGTCCCCACAGGTATGGGCAAGAGCGGCAAGTCCGGCAACGGCAGCTACCGGCT
TCTAGACCACTACAAGTACCTGACCGCTTGGTTTGAGCTGCTGAACCTGCCCAAGAAGA
TCATCTTCGTCGGCCACGACTGGGGCAGCGCCCTGGCCTTTCACTACGCCTACGAGCAC
CAGGACCGGATCAAGGCCATCGTGCACATGGAAAGCGTGGTGGACGTGATCGAGAGCTG
GATGGGCTGGCCCGACATCGAGGAAGAACTGGCCCTGATCAAGAGCGAAGAGGGCGAGA
AGATGGTGCTGGAAAACAACTTCTTCGTGGAAACCCTGCTGCCCAGCAAGATCATGCGG
AAGCTGGAACCCGAAGAGTTCGCCGCCTACCTGGAACCCTTCAAAGAAAAGGGCGAAGT
GCGGAGGCCCACCCTGAGCTGGCCCAGAGAGATCCCCCTGGTCAAGGGCGGCAAGCCCG
ACGTGGTGCAGATCGTGCGGAACTACAACGCCTACCTGCGGGCCAGCGACGACCTGCCT
AAGCTGTTCATCGAGAGCGACCCCGGCTTCTTCAGCAACGCCATCGTGGAAGGCGCCAA
GAAGTTCCCCAACACCGAGTTCGTGAAAGTGAAGGGCCTGCACTTCCTCCAGGAAGATG
CCCCCGACGAGATGGGCAAGTACATCAAGAGCTTCGTGGAACGGGTGCTGAAGAACGAG
CAGTGAGCGGCCGCAAAATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCT
AATAAAATGAGGAAATTGCATCACAACACTCAACCCTATCTCGGTCTATTCTTTTGATT
TATAAGGGATTTTGCCGATTTCGGCCTATTGGTTAAAAAATGAGCTGATTTAACAAAAA
TTTAACGCGAATTAATTCTGTGGAATGTGTGTCAGTTAGGGTGTGGAAAGTCCCCAGGC
TCCCCAGCAGGCAGAAGTATGCAAAGCATGCATCTCAATTAGTCAGCAACCAGGTGTGG
AAAGTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCGCC
CATTCTCCGCCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAGGCTTTTGCA
AAAAGCTCCCGGGAGCTTGTATATCCATTTTCGGATCTGATCAGCACGTGTTGACAATT
AATCATCGGCATAGTATATCGGCATAGTATAATACGACAAGGTGAGGAACTAAACCATG
ACCGAGTACAAGCCCACGGTGCGCCTCGCCACCCGCGACGACGTCCCCAGGGCCGTACG
CACCCTCGCCGCCGCGTTCGCCGACTACCCCGCCACGCGCCACACCGTCGATCCGGACC
GCCACATCGAGCGGGTCACCGAGCTGCAAGAACTCTTCCTCACGCGCGTCGGGCTCGAC
ATCGGCAAGGTGTGGGTCGCGGACGACGGCGCCGCGGTGGCGGTCTGGACCACGCCGGA
GAGCGTCGAAGCGGGGGCGGTGTTCGCCGAGATCGGCCCGCGCATGGCCGAGTTGAGCG
GTTCCCGGCTGGCCGCGCAGCAACAGATGGAAGGCCTCCTGGCGCCGCACCGGCCCAAG
GAGCCCGCGTGGTTCCTGGCCACCGTCGGCGTCTCGCCCGACCACCAGGGCAAGGGTCT
GGGCAGCGCCGTCGTGCTCCCCGGAGTGGAGGCGGCCGAGCGCGCCGGGGTGCCCGCCT
TCCTGGAGACATCCGCGCCCCGCAACCTCCCCTTCTACGAGCGGCTCGGCTTCACCGTC
ACCGCCGACGTCGAGGTGCCCGAAGGACCGCGCACCTGGTGCATGACCCGCAAGCCCGG
TGCCTGACACGTGCTACGAGATTTCGATTCCACCGCCGCCTTCTATGAAAGGTTGGGCT
TCGGAATCGTTTTCCGGGACGCCGGCTGGATGATCCTCCAGCGCGGGGATCTCATGCTG
GAGTTCTTCGCCCACCCCAACTTGTTTATTGCAGCTTATAATGGTTACAAATAAAGCAA
TAGCATCACAAATTTCACAAATAAAGCATTTTTTTCACTGCATTCTAGTTGTGGTTTGT
CCAAACTCATCAATGTATCTTATCATGTCTGTATACCGTCGACCTCTAGCTAGAGCTTG
GCGTAATCATGGTCATTACCAATGCTTAATCAGTGAGGCACCTATCTCAGCGATCTGTC
TATTTCGTTCATCCATAGTTGCCTGACTCCCCGTCGTGTAGATAACTACGATACGGGAG
GGCTTACCATCTGGCCCCAGCGCTGCGATGATACCGCGAGAACCACGCTCACCGGCTCC
GGATTTATCAGCAATAAACCAGCCAGCCGGAAGGGCCGAGCGCAGAAGTGGTCCTGCAA
CTTTATCCGCCTCCATCCAGTCTATTAATTGTTGCCGGGAAGCTAGAGTAAGTAGTTCG
CCAGTTAATAGTTTGCGCAACGTTGTTGCCATCGCTACAGGCATCGTGGTGTCACGCTC
GTCGTTTGGTATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGT
CATGCCATCCGTAAGATGCTTTTCTGTGACTGGTGAGTACTCAACCAAGTCATTCTGAG
AATAGTGTATGCGGCGACCGAGTTGCTCTTGCCCGGCGTCAATACGGGATAATACCGCG
CCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATATTCTTCCT
TTTTCAATATTATTGAAGCATTTATCAGGGTTATTGTCTCATGAGCGGATACATATTTG
AATGTATTTAGAAAAATAAACAAATAGGGGTCAGTGTTACAACCAATTAACCAATTCTG
AACATTATCGCGAGCCCATTTATACCTGAATATGGCTCATAACACCCCTTGCTCATGAC
CAAAATCCCTTAACGTGAGTTACGCGCGCGTCGTTCCACTGAGCGTCAGACCCCGTAGA
AAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTTCTTCTAGTGTA
GCCGTAGTTAGCCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGGATAACCGTAT
TACCGCCTTTGAGTGAGCTGATACCGCTCGCCGCAGCCGAACGACCGAGCGCAGCGAGT
CAGTGAGCGAGGAAGCGGAAGGCGAGAGTAGGGAACTGCCAGGCATCAAACTAAGCAGA
AGGCCCCTGACGGATGGCCTTTTTGCGTTTCTACAAACTCTTTCTGTGTTGTAAAACGA
CGGCCAGTCTTAAGCTCGGGCCCCCTGGGCGGTTCTGATAACGAGTAATCGTTAATCCG
CAAATAACGTAAAAACCCGCTTCGGCGGGTTTTTTTATGGGGGGAGTTTAGGGAAAGAG
CATTTGTCAGAATATTTAAGGGCGCCTGTCACTTTGCTTGATATATGAGAATTATTTAA
CCTTATAAATGAGAAAAAAGCAACGCACTTTAAATAAGATACGTTGCTTTTTCGATTGA
TGAACACCTATAATTAAACTATTCATCTATTATTTATGATTTTTTGTATATACAATATT
TCTAGTTTGTTAAAGAGAATTAAGAAAATAAATCTCGAAAATAATAAAGGGAAAATCAG
TTTTTGATATCAAAATTATACATGTCAACGATAATACAAAATATAATACAAACTATAAG
ATGTTATCAGTATTTATTATCATTTAGAATAAATTTTGTGTCGCCCTTAATTGTGAGCG
GATAACAATTACGAGCTTCATGCACAGTGGCGTTGACATTGATTATTGACTAGCATGTT
CTTTCCTGCGTTATCCCCTGATTCTGTGGATAACCGTATTACCGCCATGCATTAGTTAT
TAATAACATACGCTCTCCATCAAAACAAAACGAAACAAAACAAACTAGCAAAATAGGCT
GTCCCCAGTGCAAGTGCAGGTGCCAGAACATTTCTCT

### Plasmid DCMV-RLuc-Int Mut

Another derivative plasmid was produced having a mutation at the splice acceptor site, like the mutation in the MECP2 gene that causes Rett Syndrome in a patient. The mutation, located two nucleotides before the end of the sequence below (a replacement of C to G), is shown in Italics and is underlined.

In this case, the following mutated “intron” sequence (designated SEQ ID NO: 5) was inserted at the location indicated above:

GTTGGTATCAAGGTTACAAGACAGGTTTAAGGAGACCAATAGAAACTGGG
CATGTGGAGACAGAGAAGACTCTTGGGTTTCTGAGAGAGGCCTGGGGGGG
TCAGCGGCAGGCAGACGAGTGAGTGGCTTTGGTGACAGGTCCTCAGGGGC
AGCCAGGCAGTGTGACTCTCGTTCAATAGTAACGTTTGTCAGAGCGTTGT
CACCACCATCCGCTCTGCCCTATCTCTGACATTGCTATGGAGAGCCTCTA
ATTGTTCCTTGTGTCTTTCTGTTTGTCCCCAGAG

After the insertion the derivative plasmid termed “Plasmid pCMV-RLuc-Int_Mut” had the following sequence (designated SEQ ID NO: 6):

ACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTG
GCACCAAAATCAACGTCAGAGCGTTGTCACCACCATCCGCTCTGCCCTATCTCGGGACT
TTCCAAAATGTCGTAACAACTCCGCCCCATTGACGCAAATGGGCGGTAGGCGTGTACGG
TGGGAGGTCTATATAAGCAGAGCTCTCTGGCTAACTAGAGAACCCACTGCTTACTGGCT
TATCGAAATTAATACGACTCACTATAGGGGATCCACATTTGCTTCTGACACAACTGTGT
TCACTAGCAACCTCAAACAGACACCATGGCCAGCAAGGTGTACGACCCCGAGCAGCGGA
AGCGGATGATCACCGGCCCTCAGTGGTGGGCTCGGTGCAAGCAGATGAACGTGCTGGAC
AGCTTCATCAACTACTACGACAGCGAGAAGCACGCCGAGAACGCCGTGATCTTCCTGCA
CGGCAACGCCACCAGCAGCTACCTGTGGCGGCACGTGGTGCCCCACATCGAGCCTGTGG
CCAGATGCATCATCCCAGATCTGATAGGTTGGTATCAAGGTTACAAGACAGGTTTAAGG
AGACCAATAGAAACTGGGCATGTGGAGACAGAGAAGACTCTTGGGTTTCTGAGAGAGGC
CTGGGGGGGTCAGCGGCAGGCAGACGAGTGAGTGGCTTTGGTGACAGGTCCTCAGGGGC
AGCCAGGCAGTGTGACTCTCGTTCAATAGTAACGTTTGTCAGAGCGTTGTCACCACCAT
CCGCTCTGCCCTATCTCTGACATTGCTATGGAGAGCCTCTAATTGTTCCTTGTGTCTTT
CTGTTTGTCCCCAGAGGTATGGGCAAGAGCGGCAAGTCCGGCAACGGCAGCTACCGGCT
TCTAGACCACTACAAGTACCTGACCGCTTGGTTTGAGCTGCTGAACCTGCCCAAGAAGA
TCATCTTCGTCGGCCACGACTGGGGCAGCGCCCTGGCCTTTCACTACGCCTACGAGCAC
CAGGACCGGATCAAGGCCATCGTGCACATGGAAAGCGTGGTGGACGTGATCGAGAGCTG
GATGGGCTGGCCCGACATCGAGGAAGAACTGGCCCTGATCAAGAGCGAAGAGGGCGAGA
AGATGGTGCTGGAAAACAACTTCTTCGTGGAAACCCTGCTGCCCAGCAAGATCATGCGG
AAGCTGGAACCCGAAGAGTTCGCCGCCTACCTGGAACCCTTCAAAGAAAAGGGCGAAGT
GCGGAGGCCCACCCTGAGCTGGCCCAGAGAGATCCCCCTGGTCAAGGGCGGCAAGCCCG
ACGTGGTGCAGATCGTGCGGAACTACAACGCCTACCTGCGGGCCAGCGACGACCTGCCT
AAGCTGTTCATCGAGAGCGACCCCGGCTTCTTCAGCAACGCCATCGTGGAAGGCGCCAA
GAAGTTCCCCAACACCGAGTTCGTGAAAGTGAAGGGCCTGCACTTCCTCCAGGAAGATG
CCCCCGACGAGATGGGCAAGTACATCAAGAGCTTCGTGGAACGGGTGCTGAAGAACGAG
CAGTGAGCGGCCGCAAAATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCT
AATAAAATGAGGAAATTGCATCACAACACTCAACCCTATCTCGGTCTATTCTTTTGATT
TATAAGGGATTTTGCCGATTTCGGCCTATTGGTTAAAAAATGAGCTGATTTAACAAAAA
TTTAACGCGAATTAATTCTGTGGAATGTGTGTCAGTTAGGGTGTGGAAAGTCCCCAGGC
TCCCCAGCAGGCAGAAGTATGCAAAGCATGCATCTCAATTAGTCAGCAACCAGGTGTGG
AAAGTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCGCC
CATTCTCCGCCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAGGCTTTTGCA
AAAAGCTCCCGGGAGCTTGTATATCCATTTTCGGATCTGATCAGCACGTGTTGACAATT
AATCATCGGCATAGTATATCGGCATAGTATAATACGACAAGGTGAGGAACTAAACCATG
ACCGAGTACAAGCCCACGGTGCGCCTCGCCACCCGCGACGACGTCCCCAGGGCCGTACG
CACCCTCGCCGCCGCGTTCGCCGACTACCCCGCCACGCGCCACACCGTCGATCCGGACC
GCCACATCGAGCGGGTCACCGAGCTGCAAGAACTCTTCCTCACGCGCGTCGGGCTCGAC
ATCGGCAAGGTGTGGGTCGCGGACGACGGCGCCGCGGTGGCGGTCTGGACCACGCCGGA
GAGCGTCGAAGCGGGGGCGGTGTTCGCCGAGATCGGCCCGCGCATGGCCGAGTTGAGCG
GTTCCCGGCTGGCCGCGCAGCAACAGATGGAAGGCCTCCTGGCGCCGCACCGGCCCAAG
GAGCCCGCGTGGTTCCTGGCCACCGTCGGCGTCTCGCCCGACCACCAGGGCAAGGGTCT
GGGCAGCGCCGTCGTGCTCCCCGGAGTGGAGGCGGCCGAGCGCGCCGGGGTGCCCGCCT
TCCTGGAGACATCCGCGCCCCGCAACCTCCCCTTCTACGAGCGGCTCGGCTTCACCGTC
ACCGCCGACGTCGAGGTGCCCGAAGGACCGCGCACCTGGTGCATGACCCGCAAGCCCGG
TGCCTGACACGTGCTACGAGATTTCGATTCCACCGCCGCCTTCTATGAAAGGTTGGGCT
TCGGAATCGTTTTCCGGGACGCCGGCTGGATGATCCTCCAGCGCGGGGATCTCATGCTG
GAGTTCTTCGCCCACCCCAACTTGTTTATTGCAGCTTATAATGGTTACAAATAAAGCAA
TAGCATCACAAATTTCACAAATAAAGCATTTTTTTCACTGCATTCTAGTTGTGGTTTGT
CCAAACTCATCAATGTATCTTATCATGTCTGTATACCGTCGACCTCTAGCTAGAGCTTG
GCGTAATCATGGTCATTACCAATGCTTAATCAGTGAGGCACCTATCTCAGCGATCTGTC
TATTTCGTTCATCCATAGTTGCCTGACTCCCCGTCGTGTAGATAACTACGATACGGGAG
GGCTTACCATCTGGCCCCAGCGCTGCGATGATACCGCGAGAACCACGCTCACCGGCTCC
GGATTTATCAGCAATAAACCAGCCAGCCGGAAGGGCCGAGCGCAGAAGTGGTCCTGCAA
CTTTATCCGCCTCCATCCAGTCTATTAATTGTTGCCGGGAAGCTAGAGTAAGTAGTTCG
CCAGTTAATAGTTTGCGCAACGTTGTTGCCATCGCTACAGGCATCGTGGTGTCACGCTC
GTCGTTTGGTATGGCTTCATTCAGCTCCGGTTCCCAACGATCAAGGCGAGTTACATGAT
CCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGT
CATGCCATCCGTAAGATGCTTTTCTGTGACTGGTGAGTACTCAACCAAGTCATTCTGAG
AATAGTGTATGCGGCGACCGAGTTGCTCTTGCCCGGCGTCAATACGGGATAATACCGCG
CCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATATTCTTCCT
TTTTCAATATTATTGAAGCATTTATCAGGGTTATTGTCTCATGAGCGGATACATATTTG
AATGTATTTAGAAAAATAAACAAATAGGGGTCAGTGTTACAACCAATTAACCAATTCTG
AACATTATCGCGAGCCCATTTATACCTGAATATGGCTCATAACACCCCTTGCTCATGAC
CAAAATCCCTTAACGTGAGTTACGCGCGCGTCGTTCCACTGAGCGTCAGACCCCGTAGA
AAAGATCAAAGGATCTTCTTGAGATCCTTTTTTTCTGCGCGTAATCTGCTGCTTGCAAA
CAAAAAAACCACCGCTACCAGCGGTGGTTTGTTTGCCGGATCAAGAGCTACCAACTCTT
TTTCCGAAGGTAACTGGCTTCAGCAGAGCGCAGATACCAAATACTGTTCTTCTAGTGTA
GCCGTAGTTAGCCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCAC
ACAGCCCAGCTTGGAGCGAACGACCTACACCGAACTGAGATACCTACAGCGTGAGCTAT
GAGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTTTGC
TGGCCTTTTGCTCACATGTTCTTTCCTGCGTTATCCCCTGATTCTGTGGATAACCGTAT
TACCGCCTTTGAGTGAGCTGATACCGCTCGCCGCAGCCGAACGACCGAGCGCAGCGAGT
CAGTGAGCGAGGAAGCGGAAGGCGAGAGTAGGGAACTGCCAGGCATCAAACTAAGCAGA
AGGCCCCTGACGGATGGCCTTTTTGCGTTTCTACAAACTCTTTCTGTGTTGTAAAACGA
CGGCCAGTCTTAAGCTCGGGCCCCCTGGGCGGTTCTGATAACGAGTAATCGTTAATCCG
CAAATAACGTAAAAACCCGCTTCGGCGGGTTTTTTTATGGGGGGAGTTTAGGGAAAGAG
CATTTGTCAGAATATTTAAGGGCGCCTGTCACTTTGCTTGATATATGAGAATTATTTAA
CCTTATAAATGAGAAAAAAGCAACGCACTTTAAATAAGATACGTTGCTTTTTCGATTGA
TGAACACCTATAATTAAACTATTCATCTATTATTTATGATTTTTTGTATATACAATATT
TCTAGTTTGTTAAAGAGAATTAAGAAAATAAATCTCGAAAATAATAAAGGGAAAATCAG
TTTTTGATATCAAAATTATACATGTCAACGATAATACAAAATATAATACAAACTATAAG
ATGTTATCAGTATTTATTATCATTTAGAATAAATTTTGTGTCGCCCTTAATTGTGAGCG
GATAACAATTACGAGCTTCATGCACAGTGGCGTTGACATTGATTATTGACTAGCATGTT
CTTTCCTGCGTTATCCCCTGATTCTGTGGATAACCGTATTACCGCCATGCATTAGTTAT
TAATAACATACGCTCTCCATCAAAACAAAACGAAACAAAACAAACTAGCAAAATAGGCT
GTCCCCAGTGCAAGTGCAGGTGCCAGAACATTTCTCT

### Plasmid DCMV-RLuc-AltInt WT

Another derivative plasmid was produced by inserting the same WT intron as above (SEQ ID NO: 3) in a different location within the plasmid, mimicking more closely the beginning of the 4^{th} exon of the MECP2 gene (starting with TCC). The insertion point is shown in bold in SEQ ID NO: 7 presented below, namely:

GAGCGGCAAG--intron--TCCGGCAACG

After the insertion the derivative plasmid termed “Plasmid pCMV-RLuc-AltInt_WT” had the following sequence (designated SEQ ID NO: 7):

ACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTG
GCACCAAAATCAACGGGACTTTCCAAAATGTCGTAACAACTCCGCCCCATTGACGCAAA
TGGGCGGTAGGCGTGTACGGTGGGAGGTCTATATAAGCAGAGCTCTCTGGCTAACTAGA
GAACCCACTGCTTACTGGCTTATCGAAATTAATACGACTCACTATAGGGGATCCACATT
TGCTTCTGACACAACTGTGTTCACTAGCAACCTCAAACAGACACCATGGCCAGCAAGGT
GTACGACCCCGAGCAGCGGAAGCGGATGATCACCGGCCCTCAGTGGTGGGCTCGGTGCA
AGCAGATGAACGTGCTGGACAGCTTCATCAACTACTACGACAGCGAGAAGCACGCCGAG
AACGCCGTGATCTTCCTGCACGGCAACGCCACCAGCAGCTACCTGTGGCGGCACGTGGT
GCCCCACATCGAGCCTGTGGCCAGATGCATCATCCCAGATCTGATCGGCATGGGCAAGA
GCGGCAAGGTTGGTATCAAGGTTACAAGACAGGTTTAAGGAGACCAATAGAAACTGGGC
ATGTGGAGACAGAGAAGACTCTTGGGTTTCTGAGAGAGGCCTGGGGGGGTCAGCGGCAG
GCAGACGAGTGAGTGGCTTTGGTGACAGGTCCTCAGGGGCAGCCAGGCAGTGTGACTCT
CGTTCAATAGTAACGTTTGTCAGAGCGTTGTCACCACCATCCGCTCTGCCCTATCTCTG
ACATTGCTATGGAGAGCCTCTAATTGTTCCTTGTGTCTTTCTGTTTGTCCCCACAGTCC
GGCAACGGCAGCTACCGGCTTCTAGACCACTACAAGTACCTGACCGCTTGGTTTGAGCT
GCTGAACCTGCCCAAGAAGATCATCTTCGTCGGCCACGACTGGGGCAGCGCCCTGGCCT
TTCACTACGCCTACGAGCACCAGGACCGGATCAAGGCCATCGTGCACATGGAAAGCGTG
GTGGACGTGATCGAGAGCTGGATGGGCTGGCCCGACATCGAGGAAGAACTGGCCCTGAT
CAAGAGCGAAGAGGGCGAGAAGATGGTGCTGGAAAACAACTTCTTCGTGGAAACCCTGC
TGCCCAGCAAGATCATGCGGAAGCTGGAACCCGAAGAGTTCGCCGCCTACCTGGAACCC
TTCAAAGAAAAGGGCGAAGTGCGGAGGCCCACCCTGAGCTGGCCCAGAGAGATCCCCCT
GGTCAAGGGCGGCAAGCCCGACGTGGTGCAGATCGTGCGGAACTACAACGCCTACCTGC
GGGCCAGCGACGACCTGCCTAAGCTGTTCATCGAGAGCGACCCCGGCTTCTTCAGCAAC
GCCATCGTGGAAGGCGCCAAGAAGTTCCCCAACACCGAGTTCGTGAAAGTGAAGGGCCT
GCACTTCCTCCAGGAAGATGCCCCCGACGAGATGGGCAAGTACATCAAGAGCTTCGTGG
AACGGGTGCTGAAGAACGAGCAGTGAGCGGCCGCAAAATCAGCCTCGACTGTGCCTTCT
AGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCACAACACTCAACCCTAT
CTCGGTCTATTCTTTTGATTTATAAGGGATTTTGCCGATTTCGGCCTATTGGTTAAAAA
ATGAGCTGATTTAACAAAAATTTAACGCGAATTAATTCTGTGGAATGTGTGTCAGTTAG
GGTGTGGAAAGTCCCCAGGCTCCCCAGCAGGCAGAAGTATGCAAAGCATGCATCTCAAT
TAGTCAGCAACCAGGTGTGGAAAGTCCCCAGGCTCCCCAGCAGGCAGAAGTATGCAAAG
CATGCATCTCAATTAGTCAGCAACCATAGTCCCGCCCCTAACTCCGCCCATCCCGCCCC
TAACTCCGCCCAGTTCCGCCCATTCTCCGCCCCATGGCTGACTAATTTTTTTTATTTAT
GCAGAGGCCGAGGCCGCCTCTGCCTCTGAGCTATTCCAGAAGTAGTGAGGAGGCTTTTT
TGGAGGCCTAGGCTTTTGCAAAAAGCTCCCGGGAGCTTGTATATCCATTTTCGGATCTG
ATCAGCACGTGTTGACAATTAATCATCGGCATAGTATATCGGCATAGTATAATACGACA
AGGTGAGGAACTAAACCATGACCGAGTACAAGCCCACGGTGCGCCTCGCCACCCGCGAC
GACGTCCCCAGGGCCGTACGCACCCTCGCCGCCGCGTTCGCCGACTACCCCGCCACGCG
CCACACCGTCGATCCGGACCGCCACATCGAGCGGGTCACCGAGCTGCAAGAACTCTTCC
TCACGCGCGTCGGGCTCGACATCGGCAAGGTGTGGGTCGCGGACGACGGCGCCGCGGTG
GCGGTCTGGACCACGCCGGAGAGCGTCGAAGCGGGGGCGGTGTTCGCCGAGATCGGCCC
GCGCATGGCCGAGTTGAGCGGTTCCCGGCTGGCCGCGCAGCAACAGATGGAAGGCCTCC
TGGCGCCGCACCGGCCCAAGGAGCCCGCGTGGTTCCTGGCCACCGTCGGCGTCTCGCCC
GACCACCAGGGCAAGGGTCTGGGCAGCGCCGTCGTGCTCCCCGGAGTGGAGGCGGCCGA
GCGCGCCGGGGTGCCCGCCTTCCTGGAGACATCCGCGCCCCGCAACCTCCCCTTCTACG
AGCGGCTCGGCTTCACCGTCACCGCCGACGTCGAGGTGCCCGAAGGACCGCGCACCTGG
TGCATGACCCGCAAGCCCGGTGCCTGACACGTGCTACGAGATTTCGATTCCACCGCCGC
CTTCTATGAAAGGTTGGGCTTCGGAATCGTTTTCCGGGACGCCGGCTGGATGATCCTCC
AGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGT
CGACCTCTAGCTAGAGCTTGGCGTAATCATGGTCATTACCAATGCTTAATCAGTGAGGC
ACCTATCTCAGCGATCTGTCTATTTCGTTCATCCATAGTTGCCTGACTCCCCGTCGTGT
AGATAACTACGATACGGGAGGGCTTACCATCTGGCCCCAGCGCTGCGATGATACCGCGA
GAACCACGCTCACCGGCTCCGGATTTATCAGCAATAAACCAGCCAGCCGGAAGGGCCGA
GCGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATCGCTACA
GGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATGTA
ACCCACTCGTGCACCCAACTGATCTTCAGCATCTTTTACTTTCACCAGCGTTTCTGGGT
GAGCAAAAACAGGAAGGCAAAATGCCGCAAAAAAGGGAATAAGGGCGACACGGAAATGT
TGAATACTCATATTCTTCCTTTTTCAATATTATTGAAGCATTTATCAGGGTTATTGTCT
CATGAGCGGATACATATTTGAATGTATTTAGAAAAATAAACAAATAGGGGTCAGTGTTA
CAACCAATTAACCAATTCTGAACATTATCGCGAGCCCATTTATACCTGAATATGGCTCA
TAACACCCCTTGCTCATGACCAAAATCCCTTAACGTGAGTTACGCGCGCGTCGTTCCAC
TGAGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCA
AATACTGTTCTTCTAGTGTAGCCGTAGTTAGCCCACCACTTCAAGAACTCTGTAGCACC
GCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGCCG
AACGACCGAGCGCAGCGAGTCAGTGAGCGAGGAAGCGGAAGGCGAGAGTAGGGAACTGC
CAGGCATCAAACTAAGCAGAAGGCCCCTGACGGATGGCCTTTTTGCGTTTCTACAAACT
CTTTCTGTGTTGTAAAACGACGGCCAGTCTTAAGCTCGGGCCCCCTGGGCGGTTCTGAT
AACGAGTAATCGTTAATCCGCAAATAACGTAAAAACCCGCTTCGGCGGGTTTTTTTATG
GGGGGAGTTTAGGGAAAGAGCATTTGTCAGAATATTTAAGGGCGCCTGTCACTTTGCTT
GATATATGAGAATTATTTAACCTTATAAATGAGAAAAAAGCAACGCACTTTAAATAAGA
TACGTTGCTTTTTCGATTGATGAACACCTATAATTAAACTATTCATCTATTATTTATGA
TTTTTTGTATATACAATATTTCTAGTTTGTTAAAGAGAATTAAGAAAATAAATCTCGAA
AATAATAAAGGGAAAATCAGTTTTTGATATCAAAATTATACATGTCAACGATAATACAA
AATATAATACAAACTATAAGATGTTATCAGTATTTATTATCATTTAGAATAAATTTTGT
GTCGCCCTTAATTGTGAGCGGATAACAATTACGAGCTTCATGCACAGTGGCGTTGACAT
TGATTATTGACTAGCATGTTCTTTCCTGCGTTATCCCCTGATTCTGTGGATAACCGTAT
TACCGCCATGCATTAGTTATTAATAACATACGCTCTCCATCAAAACAAAACGAAACAAA
ACAAACTAGCAAAATAGGCTGTCCCCAGTGCAAGTGCAGGTGCCAGAACATTTCTCT

### Plasmid DCMV-RLuc-AltInt Mut

This derivative plasmid is like the alternative plasmid described above but comprises the mutated intron insertion (SEQ ID NO: 5) instead of the WT intron. Namely, in this plasmid the intron is identical to that of pCMV-RLuc-Int_Mut, containing the mutant splice acceptor site, but it was inserted at the alternative site.

After the insertion the derivative plasmid termed “Plasmid pCMV-RLuc-AltInt_Mut” had the following sequence (designated SEQ ID NO: 8):

ACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTG
GCACCAAAATCAACGGGACTTTCCAAAATGTCGTAACAACTCCGCCCCATTGACGCAAA
TGGGCGGTAGGCGTGTACGGTGGGAGGTCTATATAAGCAGAGCTCTCTGGCTAACTAGA
GAACCCACTGCTTACTGGCTTATCGAAATTAATACGACTCACTATAGGGGATCCACATT
TGCTTCTGACACAACTGTGTTCACTAGCAACCTCAAACAGACACCATGGCCAGCAAGGT
GTACGACCCCGAGCAGCGGAAGCGGATGATCACCGGCCCTCAGTGGTGGGCTCGGTGCA
AGCAGATGAACGTGCTGGACAGCTTCATCAACTACTACGACAGCGAGAAGCACGCCGAG
AACGCCGTGATCTTCCTGCACGGCAACGCCACCAGCAGCTACCTGTGGCGGCACGTGGT
GCCCCACATCGAGCCTGTGGCCAGATGCATCATCCCAGATCTGATCGGCATGGGCAAGA
GCGGCAAGGTTGGTATCAAGGTTACAAGACAGGTTTAAGGAGACCAATAGAAACTGGGC
ATGTGGAGACAGAGAAGACTCTTGGGTTTCTGAGAGAGGCCTGGGGGGGTCAGCGGCAG
GCAGACGAGTGAGTGGCTTTGGTGACAGGTCCTCAGGGGCAGCCAGGCAGTGTGACTCT
CGTTCAATAGTAACGTTTGTCAGAGCGTTGTCACCACCATCCGCTCTGCCCTATCTCTG
ACATTGCTATGGAGAGCCTCTAATTGTTCCTTGTGTCTTTCTGTTTGTCCCCAGAGTCC
GGCAACGGCAGCTACCGGCTTCTAGACCACTACAAGTACCTGACCGCTTGGTTTGAGCT
GCTGAACCTGCCCAAGAAGATCATCTTCGTCGGCCACGACTGGGGCAGCGCCCTGGCCT
TTCACTACGCCTACGAGCACCAGGACCGGATCAAGGCCATCGTGCACATGGAAAGCGTG
GTGGACGTGATCGAGAGCTGGATGGGCTGGCCCGACATCGAGGAAGAACTGGCCCTGAT
CAAGAGCGAAGAGGGCGAGAAGATGGTGCTGGAAAACAACTTCTTCGTGGAAACCCTGC
TGCCCAGCAAGATCATGCGGAAGCTGGAACCCGAAGAGTTCGCCGCCTACCTGGAACCC
TTCAAAGAAAAGGGCGAAGTGCGGAGGCCCACCCTGAGCTGGCCCAGAGAGATCCCCCT
GGTCAAGGGCGGCAAGCCCGACGTGGTGCAGATCGTGCGGAACTACAACGCCTACCTGC
GGGCCAGCGACGACCTGCCTAAGCTGTTCATCGAGAGCGACCCCGGCTTCTTCAGCAAC
GCCATCGTGGAAGGCGCCAAGAAGTTCCCCAACACCGAGTTCGTGAAAGTGAAGGGCCT
GCACTTCCTCCAGGAAGATGCCCCCGACGAGATGGGCAAGTACATCAAGAGCTTCGTGG
AACGGGTGCTGAAGAACGAGCAGTGAGCGGCCGCAAAATCAGCCTCGACTGTGCCTTCT
AGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCACAACACTCAACCCTAT
CTCGGTCTATTCTTTTGATTTATAAGGGATTTTGCCGATTTCGGCCTATTGGTTAAAAA
ATGAGCTGATTTAACAAAAATTTAACGCGAATTAATTCTGTGGAATGTGTGTCAGTTAG
GGTGTGGAAAGTCCCCAGGCTCCCCAGCAGGCAGAAGTATGCAAAGCATGCATCTCAAT
TAGTCAGCAACCAGGTGTGGAAAGTCCCCAGGCTCCCCAGCAGGCAGAAGTATGCAAAG
CATGCATCTCAATTAGTCAGCAACCATAGTCCCGCCCCTAACTCCGCCCATCCCGCCCC
TAACTCCGCCCAGTTCCGCCCATTCTCCGCCCCATGGCTGACTAATTTTTTTTATTTAT
GCAGAGGCCGAGGCCGCCTCTGCCTCTGAGCTATTCCAGAAGTAGTGAGGAGGCTTTTT
TGGAGGCCTAGGCTTTTGCAAAAAGCTCCCGGGAGCTTGTATATCCATTTTCGGATCTG
ATCAGCACGTGTTGACAATTAATCATCGGCATAGTATATCGGCATAGTATAATACGACA
AGGTGAGGAACTAAACCATGACCGAGTACAAGCCCACGGTGCGCCTCGCCACCCGCGAC
GACGTCCCCAGGGCCGTACGCACCCTCGCCGCCGCGTTCGCCGACTACCCCGCCACGCG
CCACACCGTCGATCCGGACCGCCACATCGAGCGGGTCACCGAGCTGCAAGAACTCTTCC
TCACGCGCGTCGGGCTCGACATCGGCAAGGTGTGGGTCGCGGACGACGGCGCCGCGGTG
GCGGTCTGGACCACGCCGGAGAGCGTCGAAGCGGGGGCGGTGTTCGCCGAGATCGGCCC
GCGCATGGCCGAGTTGAGCGGTTCCCGGCTGGCCGCGCAGCAACAGATGGAAGGCCTCC
TGGCGCCGCACCGGCCCAAGGAGCCCGCGTGGTTCCTGGCCACCGTCGGCGTCTCGCCC
GACCACCAGGGCAAGGGTCTGGGCAGCGCCGTCGTGCTCCCCGGAGTGGAGGCGGCCGA
GCGCGCCGGGGTGCCCGCCTTCCTGGAGACATCCGCGCCCCGCAACCTCCCCTTCTACG
AGCGGCTCGGCTTCACCGTCACCGCCGACGTCGAGGTGCCCGAAGGACCGCGCACCTGG
TGCATGACCCGCAAGCCCGGTGCCTGACACGTGCTACGAGATTTCGATTCCACCGCCGC
CTTCTATGAAAGGTTGGGCTTCGGAATCGTTTTCCGGGACGCCGGCTGGATGATCCTCC
AGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGT
CGACCTCTAGCTAGAGCTTGGCGTAATCATGGTCATTACCAATGCTTAATCAGTGAGGC
ACCTATCTCAGCGATCTGTCTATTTCGTTCATCCATAGTTGCCTGACTCCCCGTCGTGT
AGATAACTACGATACGGGAGGGCTTACCATCTGGCCCCAGCGCTGCGATGATACCGCGA
GAACCACGCTCACCGGCTCCGGATTTATCAGCAATAAACCAGCCAGCCGGAAGGGCCGA
GCGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATCGCTACA
GGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATGTA
ACCCACTCGTGCACCCAACTGATCTTCAGCATCTTTTACTTTCACCAGCGTTTCTGGGT
GAGCAAAAACAGGAAGGCAAAATGCCGCAAAAAAGGGAATAAGGGCGACACGGAAATGT
TGAATACTCATATTCTTCCTTTTTCAATATTATTGAAGCATTTATCAGGGTTATTGTCT
CATGAGCGGATACATATTTGAATGTATTTAGAAAAATAAACAAATAGGGGTCAGTGTTA
CAACCAATTAACCAATTCTGAACATTATCGCGAGCCCATTTATACCTGAATATGGCTCA
TAACACCCCTTGCTCATGACCAAAATCCCTTAACGTGAGTTACGCGCGCGTCGTTCCAC
TGAGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCA
AATACTGTTCTTCTAGTGTAGCCGTAGTTAGCCCACCACTTCAAGAACTCTGTAGCACC
GCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGCCG
AACGACCGAGCGCAGCGAGTCAGTGAGCGAGGAAGCGGAAGGCGAGAGTAGGGAACTGC
CAGGCATCAAACTAAGCAGAAGGCCCCTGACGGATGGCCTTTTTGCGTTTCTACAAACT
CTTTCTGTGTTGTAAAACGACGGCCAGTCTTAAGCTCGGGCCCCCTGGGCGGTTCTGAT
AACGAGTAATCGTTAATCCGCAAATAACGTAAAAACCCGCTTCGGCGGGTTTTTTTATG
GGGGGAGTTTAGGGAAAGAGCATTTGTCAGAATATTTAAGGGCGCCTGTCACTTTGCTT
GATATATGAGAATTATTTAACCTTATAAATGAGAAAAAAGCAACGCACTTTAAATAAGA
TACGTTGCTTTTTCGATTGATGAACACCTATAATTAAACTATTCATCTATTATTTATGA
TTTTTTGTATATACAATATTTCTAGTTTGTTAAAGAGAATTAAGAAAATAAATCTCGAA
AATAATAAAGGGAAAATCAGTTTTTGATATCAAAATTATACATGTCAACGATAATACAA
AATATAATACAAACTATAAGATGTTATCAGTATTTATTATCATTTAGAATAAATTTTGT
GTCGCCCTTAATTGTGAGCGGATAACAATTACGAGCTTCATGCACAGTGGCGTTGACAT
TGATTATTGACTAGCATGTTCTTTCCTGCGTTATCCCCTGATTCTGTGGATAACCGTAT
TACCGCCATGCATTAGTTATTAATAACATACGCTCTCCATCAAAACAAAACGAAACAAA
ACAAACTAGCAAAATAGGCTGTCCCCAGTGCAAGTGCAGGTGCCAGAACATTTCTCT

### Plasmid DCMV-RLuc-AltInt_WTBPMut

Finally, an additional derivative plasmid was produced. This derivative plasmid contained the WT intron sequence in which the splicing branch point signal sequence was mutated to a sequence that is not recognized as a branch point (shown in bold underlined). This intron sequence is designated SEQ ID NO: 9:

GTTGGTATCAAGGTTACAAGACAGGTTTAAGGAGACCAATAGAAACTGG
GCATGTGGAGACAGAGAAGACTCTTGGGTTTCTGAGAGAGGCCTGGGGG
GGTCAGCGGCAGGCAGACGAGTGAGTGGCTTTGGTGACAGGTCCTCAGG
GGCAGCCAGGCAGTGTGACTCTCGTTCAATAGTAACGTTTGTCAGAGCG
TTGTCACCACCATCCGCTCTGCCCTATCTCTGACATTGCTATGGAGAGC
CTGCCTGTGTTCCTTGTGTCTTTCTGTTTGTCCCCACAG

After the insertion the derivative plasmid termed “Plasmid pCMV-RLuc-AltInt_WTBPMut” had the following sequence (designated SEQ ID NO: 10):

ACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAGTG
TATCATATGCCAAGTACGCCCCCTATTGACGTCAATGACGGTAAATGGCCCGCCTGGCA
TTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGG
TTTGACTCACGGGGATTTCCAAGTCTCCACCCCATTGACGTCAATGGGAGTTTGTTTTG
GCACCAAAATCAACGGGACTTTCCAAAATGTCGTAACAACTCCGCCCCATTGACGCAAA
TGGGCGGTAGGCGTGTACGGTGGGAGGTCTATATAAGCAGAGCTCTCTGGCTAACTAGA
GAACCCACTGCTTACTGGCTTATCGAAATTAATACGACTCACTATAGGGGATCCACATT
TGCTTCTGACACAACTGTGTTCACTAGCAACCTCAAACAGACACCATGGCCAGCAAGGT
GTACGACCCCGAGCAGCGGAAGCGGATGATCACCGGCCCTCAGTGGTGGGCTCGGTGCA
AGCAGATGAACGTGCTGGACAGCTTCATCAACTACTACGACAGCGAGAAGCACGCCGAG
AACGCCGTGATCTTCCTGCACGGCAACGCCACCAGCAGCTACCTGTGGCGGCACGTGGT
GCCCCACATCGAGCCTGTGGCCAGATGCATCATCCCAGATCTGATCGGCATGGGCAAGA
GCGGCAAGGTTGGTATCAAGGTTACAAGACAGGTTTAAGGAGACCAATAGAAACTGGGC
ATGTGGAGACAGAGAAGACTCTTGGGTTTCTGAGAGAGGCCTGGGGGGGTCAGCGGCAG
GCAGACGAGTGAGTGGCTTTGGTGACAGGTCCTCAGGGGCAGCCAGGCAGTGTGACTCT
CGTTCAATAGTAACGTTTGTCAGAGCGTTGTCACCACCATCCGCTCTGCCCTATCTCTG
ACATTGCTATGGAGAGCCTGCCTGTGTTCCTTGTGTCTTTCTGTTTGTCCCCACAGTCC
GGCAACGGCAGCTACCGGCTTCTAGACCACTACAAGTACCTGACCGCTTGGTTTGAGCT
GCTGAACCTGCCCAAGAAGATCATCTTCGTCGGCCACGACTGGGGCAGCGCCCTGGCCT
TTCACTACGCCTACGAGCACCAGGACCGGATCAAGGCCATCGTGCACATGGAAAGCGTG
GTGGACGTGATCGAGAGCTGGATGGGCTGGCCCGACATCGAGGAAGAACTGGCCCTGAT
CAAGAGCGAAGAGGGCGAGAAGATGGTGCTGGAAAACAACTTCTTCGTGGAAACCCTGC
TGCCCAGCAAGATCATGCGGAAGCTGGAACCCGAAGAGTTCGCCGCCTACCTGGAACCC
TTCAAAGAAAAGGGCGAAGTGCGGAGGCCCACCCTGAGCTGGCCCAGAGAGATCCCCCT
GGTCAAGGGCGGCAAGCCCGACGTGGTGCAGATCGTGCGGAACTACAACGCCTACCTGC
GGGCCAGCGACGACCTGCCTAAGCTGTTCATCGAGAGCGACCCCGGCTTCTTCAGCAAC
GCCATCGTGGAAGGCGCCAAGAAGTTCCCCAACACCGAGTTCGTGAAAGTGAAGGGCCT
GCACTTCCTCCAGGAAGATGCCCCCGACGAGATGGGCAAGTACATCAAGAGCTTCGTGG
AACGGGTGCTGAAGAACGAGCAGTGAGCGGCCGCAAAATCAGCCTCGACTGTGCCTTCT
AGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCACAACACTCAACCCTAT
CTCGGTCTATTCTTTTGATTTATAAGGGATTTTGCCGATTTCGGCCTATTGGTTAAAAA
ATGAGCTGATTTAACAAAAATTTAACGCGAATTAATTCTGTGGAATGTGTGTCAGTTAG
GGTGTGGAAAGTCCCCAGGCTCCCCAGCAGGCAGAAGTATGCAAAGCATGCATCTCAAT
TAGTCAGCAACCAGGTGTGGAAAGTCCCCAGGCTCCCCAGCAGGCAGAAGTATGCAAAG
CATGCATCTCAATTAGTCAGCAACCATAGTCCCGCCCCTAACTCCGCCCATCCCGCCCC
TAACTCCGCCCAGTTCCGCCCATTCTCCGCCCCATGGCTGACTAATTTTTTTTATTTAT
GCAGAGGCCGAGGCCGCCTCTGCCTCTGAGCTATTCCAGAAGTAGTGAGGAGGCTTTTT
TGGAGGCCTAGGCTTTTGCAAAAAGCTCCCGGGAGCTTGTATATCCATTTTCGGATCTG
ATCAGCACGTGTTGACAATTAATCATCGGCATAGTATATCGGCATAGTATAATACGACA
AGGTGAGGAACTAAACCATGACCGAGTACAAGCCCACGGTGCGCCTCGCCACCCGCGAC
GACGTCCCCAGGGCCGTACGCACCCTCGCCGCCGCGTTCGCCGACTACCCCGCCACGCG
CCACACCGTCGATCCGGACCGCCACATCGAGCGGGTCACCGAGCTGCAAGAACTCTTCC
TCACGCGCGTCGGGCTCGACATCGGCAAGGTGTGGGTCGCGGACGACGGCGCCGCGGTG
GCGGTCTGGACCACGCCGGAGAGCGTCGAAGCGGGGGCGGTGTTCGCCGAGATCGGCCC
GCGCATGGCCGAGTTGAGCGGTTCCCGGCTGGCCGCGCAGCAACAGATGGAAGGCCTCC
TGGCGCCGCACCGGCCCAAGGAGCCCGCGTGGTTCCTGGCCACCGTCGGCGTCTCGCCC
GACCACCAGGGCAAGGGTCTGGGCAGCGCCGTCGTGCTCCCCGGAGTGGAGGCGGCCGA
GCGCGCCGGGGTGCCCGCCTTCCTGGAGACATCCGCGCCCCGCAACCTCCCCTTCTACG
AGCGGCTCGGCTTCACCGTCACCGCCGACGTCGAGGTGCCCGAAGGACCGCGCACCTGG
TGCATGACCCGCAAGCCCGGTGCCTGACACGTGCTACGAGATTTCGATTCCACCGCCGC
CTTCTATGAAAGGTTGGGCTTCGGAATCGTTTTCCGGGACGCCGGCTGGATGATCCTCC
AGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACT
GCATTCTAGTTGTGGTTTGTCCAAACTCATCAATGTATCTTATCATGTCTGTATACCGT
CGACCTCTAGCTAGAGCTTGGCGTAATCATGGTCATTACCAATGCTTAATCAGTGAGGC
ACCTATCTCAGCGATCTGTCTATTTCGTTCATCCATAGTTGCCTGACTCCCCGTCGTGT
AGATAACTACGATACGGGAGGGCTTACCATCTGGCCCCAGCGCTGCGATGATACCGCGA
GAACCACGCTCACCGGCTCCGGATTTATCAGCAATAAACCAGCCAGCCGGAAGGGCCGA
GCGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATCGCTACA
GGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTC
CTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTA
CTCAACCAAGTCATTCTGAGAATAGTGTATGCGGCGACCGAGTTGCTCTTGCCCGGCGT
CAATACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATGTA
ACCCACTCGTGCACCCAACTGATCTTCAGCATCTTTTACTTTCACCAGCGTTTCTGGGT
GAGCAAAAACAGGAAGGCAAAATGCCGCAAAAAAGGGAATAAGGGCGACACGGAAATGT
TGAATACTCATATTCTTCCTTTTTCAATATTATTGAAGCATTTATCAGGGTTATTGTCT
CATGAGCGGATACATATTTGAATGTATTTAGAAAAATAAACAAATAGGGGTCAGTGTTA
CAACCAATTAACCAATTCTGAACATTATCGCGAGCCCATTTATACCTGAATATGGCTCA
TAACACCCCTTGCTCATGACCAAAATCCCTTAACGTGAGTTACGCGCGCGTCGTTCCAC
TGAGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGATACCA
AATACTGTTCTTCTAGTGTAGCCGTAGTTAGCCCACCACTTCAAGAACTCTGTAGCACC
GCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGC
TGAACGGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACA
GGTATCCGGTAAGCGGCAGGGTCGGAACAGGAGAGCGCACGAGGGAGCTTCCAGGGGGA
AACGCCTGGTATCTTTATAGTCCTGTCGGGTTTCGCCACCTCTGACTTGAGCGTCGATT
TTTGTGATGCTCGTCAGGGGGGCGGAGCCTATGGAAAAACGCCAGCAACGCGGCCTTTT
TACGGTTCCTGGCCTTTTGCTGGCCTTTTGCTCACATGTTCTTTCCTGCGTTATCCCCT
GATTCTGTGGATAACCGTATTACCGCCTTTGAGTGAGCTGATACCGCTCGCCGCAGCCG
AACGACCGAGCGCAGCGAGTCAGTGAGCGAGGAAGCGGAAGGCGAGAGTAGGGAACTGC
CAGGCATCAAACTAAGCAGAAGGCCCCTGACGGATGGCCTTTTTGCGTTTCTACAAACT
CTTTCTGTGTTGTAAAACGACGGCCAGTCTTAAGCTCGGGCCCCCTGGGCGGTTCTGAT
AACGAGTAATCGTTAATCCGCAAATAACGTAAAAACCCGCTTCGGCGGGTTTTTTTATG
GGGGGAGTTTAGGGAAAGAGCATTTGTCAGAATATTTAAGGGCGCCTGTCACTTTGCTT
GATATATGAGAATTATTTAACCTTATAAATGAGAAAAAAGCAACGCACTTTAAATAAGA
TACGTTGCTTTTTCGATTGATGAACACCTATAATTAAACTATTCATCTATTATTTATGA
TTTTTTGTATATACAATATTTCTAGTTTGTTAAAGAGAATTAAGAAAATAAATCTCGAA
AATAATAAAGGGAAAATCAGTTTTTGATATCAAAATTATACATGTCAACGATAATACAA
AATATAATACAAACTATAAGATGTTATCAGTATTTATTATCATTTAGAATAAATTTTGT
GTCGCCCTTAATTGTGAGCGGATAACAATTACGAGCTTCATGCACAGTGGCGTTGACAT
TGATTATTGACTAGCATGTTCTTTCCTGCGTTATCCCCTGATTCTGTGGATAACCGTAT
TACCGCCATGCATTAGTTATTAATAACATACGCTCTCCATCAAAACAAAACGAAACAAA
ACAAACTAGCAAAATAGGCTGTCCCCAGTGCAAGTGCAGGTGCCAGAACATTTCTCT

### Example 3: Production of Ocirc RNA Oligonucleotides

All RNA oligonucleotides were synthetized by IDT.

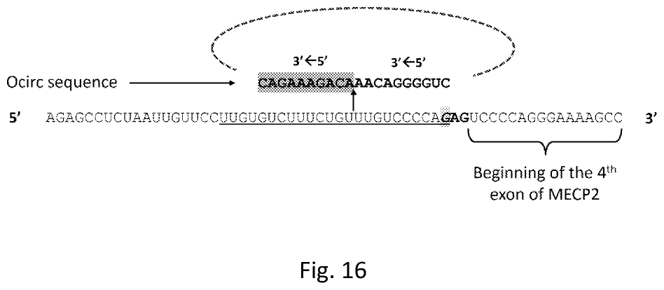

Regular RNA oligonucleotides were designed to match a specific sequence in the 3′ region of the human MECP2 3^{rd} intron. FIG. 16, is a schematic representation showing the structure of the RNA oligonucleotides after binding. The beginning of the 4^{th} exon of MECP2 is indicated. The splice acceptor is shown in bold letters (GAG) with the mutated nucleotide (G instead of C) shown in Italics. The Ocirc sequence is shown on top: the highlighted sequence is the 5′ antisense arm and the non-highlighted sequence is the 3′ antisense arm. The dashed line represents the RNA sequence found in between the arms of the Ocirc molecule. In general, it can be any sequence of choice. In this specific example, it has a PPT sequence and is designed to bind the spliceosome proteins. The black line marks the contact region of the two arms of the Ocirc on the template of the MECP2 sequence. The underlined area is the PPT sequence of the human MECP2 gene. The Ocirc sequence becomes attached to it by base-pairing.

The following oligonucleotides were used (the uracil nucleotides (U) were replaced with thymine nucleotides (T) in the sequence listing):

Ocirc-temp (designated SEQ ID NO: 11):
5′-AGAGCCUCUAAUUGUUCCUUGUGUCUUUCUGUUUGUCCCCAGAGUC
CCCAUGGAAAAGCC 3′
Ocirc-1 (designated SEQ ID NO: 12):
5′-ACAGAAAGACGCCCCCUUAUUCGUCCCCGCCUGGGGACAA-3′
Ocirc-2 (designated SEQ ID NO: 13):
5′-ACAGAAAGACCGCCCCCUUAUUCGUCCCCG[3′-3′-C-5′-5′]
UGGGGACAA-3′
Ocirc-3 (designated SEQ ID NO: 14):
5′-AAACAGAAAGCCCCCUUAUUCGUCCCCCAGCUCUGGGGAC-3′
Ocirc-4 (designated SEQ ID NO: 15):
5′-AGAAAGACACAAUCUCUGCCUAGCCCCCUUAUUCGUCCCCGCCUGG
GGACAAAC-3′
Ocirc-5 (designated SEQ ID NO: 16):
5′-AGAAAGACACAAUCUCUGCCUACGCCCCCUUAUUCGUCCCCG
[3′-3′-C-5′-5′]UGGGGACAAAC-3′
Ocirc-6 (designated SEQ ID NO: 17):
5′-ACAGAAAGACACUCUCUGCCUACCCCCUUAUUCGUCCCCCAGCUCU
GGGGACAA-3′
Ocirc-7 (designated SEQ ID NO: 18):
5′-GACAAACAGAAAGACGCCCCCUUAUUCGUCCCCGCCUGGG-3′
Ocirc-8 (designated SEQ ID NO: 19):
5′-GACAAACAGAAAGACACAAUCUCUGCCUAGCCCCCUUAUUCGUCCC
CGCCUGGG-3′
Ocirc-Cont (designated SEQ ID NO: 20):
5′-GCUCGUCACAGGCCCCCUUAUUCGUCCCCGCUAGCAGCGAU-3′

### Example 4: In-Vivo Binding of Ocirc Oligonucleotides to an RNA Template

To test whether Ocirc RNA oligonucleotides can bind to the template (Ocirc-Temp), Ocirc oligonucleotides were each mixed with the template, using concentrations as described in Table 1 below.

TABLE 1
Hybridization of RNA oligonucleotides

| | | Ocirc-Temp. | Ocirc conc. | Final Conc. |
|---|---|---|---|---|
| sample # | description | conc. μg/μl | μg/μl | (μg/μl) |
| sample #1 | Ocirc-Temp | 0.3811 | — | 0.191 |
| sample #2 | Ocirc-1 | — | 0.2557 | 0.128 |
| sample #3 | Ocirc-4 | — | 0.3439 | 0.172 |
| sample #4 | Ocirc-Temp + Ocirc-1 | 0.3811 | 0.2557 | 0.191 |
| sample #5 | Ocirc-Temp + Ocirc-4 | 0.3811 | 0.3439 | 0.191 |

The stock concentration for each of the tested oligonucleotides was 20 μM, and the final concentration was 10 μM. The final volume of the reaction was 40 μl comprised of 20 μl of the oligonucleotides+20 μl hybridization buffer (2 mM MgCl₂ in phosphate buffered saline (PBS) (PBS is 137 mM NaCl, 2.7 mM KCl, 10 mM Na₂HPO₄, and 1.8 mM KH₂PO₄) for samples 1-3 and comprised of 20 μl of each of the oligonucleotides in samples 4 and 5.

The samples were incubated at 70° C. for 5 minutes, cooled slowly (over 30 minutes) to room temperature, and placed on ice. The samples were then prepared for running on an acrylamide gel by adding a running buffer (50 μl SBx2—0.025 M Tris, 0.192 M glycine pH: 8.3). For each sample, the amount loaded was 0.15 μg/lane, at a volume of 20 μl/lane. The loading sample concentration was 0.008 μg/μl, and the final volume was 100 μl, as detailed in Table 2 below.

TABLE 2
Preparation of the samples for gel loading (Ocirc 1 and Ocirc 4)

| | | Conc. | H. |
|---|---|---|---|
| sample # | description | μg/μl | Buffer μl |
| sample #1 | Ocirc-Temp | 0.191 | 46.1 |
| sample #2 | Ocirc-1 | 0.128 | 44.1 |
| sample #3 | Ocirc-4 | 0.172 | 45.6 |
| sample #4 | Ocirc-Temp + Ocirc-1 | 0.191 | 46.1 |
| sample #5 | Ocirc-Temp + Ocirc-4 | 0.191 | 46.1 |

The samples were loaded onto an acrylamide gel and resolved by running under standard conditions.

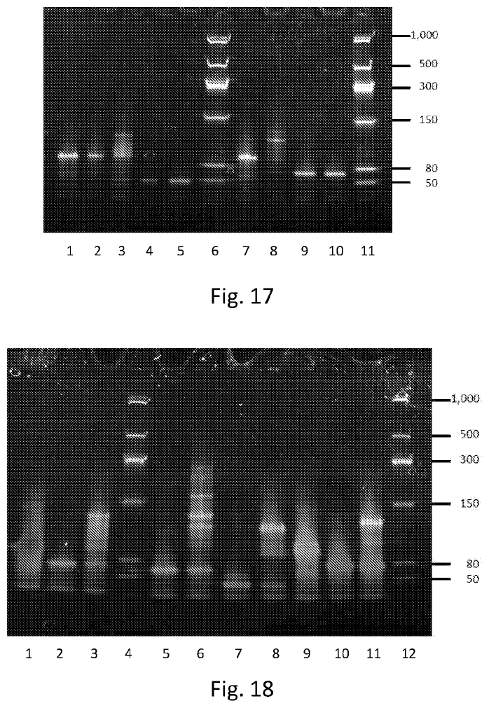

As shown in FIG. 17, mixing of the template RNA (Ocirc-temp) with Ocirc 1 (see lane 3—Ocirc template+Ocirc 1) or Ocirc 4 (see lane 8—Ocirc template+Ocirc 4) resulted in slower migrating material indicative of the binding of Ocirc 1, and Ocirc 4, to the template RNA. See also FIGS. 19A and 19B.

A similar experiment, under the same conditions, was performed with Ocirc 5, 6, 7 and 8, as detailed in Table 3 below.

TABLE 3
Preparation of the samples for gel loading (Ocirc 5-8)

| | | Ocirc-Temp. | Ocirc conc. | Final Conc. |
|---|---|---|---|---|
| sample # | description | conc. μg/μl | μg/μl | (μg/μl) |
| sample #1 | Ocirc-Temp | 0.3811 | — | 0.191 |
| sample #2 | Ocirc-5 | — | 0.5082 | 0.254 |
| sample #3 | Ocirc-6 | — | 0.5131 | 0.257 |
| sample #4 | Ocirc-7 | — | 0.3845 | 0.192 |
| sample #5 | Ocirc-8 | — | 0.5132 | 0.257 |
| sample #6 | Ocirc-control | | 0.3881 | |
| sample #7 | Ocirc-Temp + Ocirc-5 | 0.3811 | 0.5082 | 0.191 |
| sample #8 | Ocirc-Temp + Ocirc-6 | 0.3811 | 0.5131 | 0.191 |
| sample #9 | Ocirc-Temp + Ocirc-7 | 0.3811 | 0.3845 | 0.191 |
| sample #10 | Ocirc-Temp + Ocirc-8 | 0.3811 | 0.5132 | 0.191 |
| sample #11 | Ocirc-Temp + | 0.3811 | 0.3881 | 0.191 |
| | Ocirc-control | | | |

As shown in FIG. 18, mixing ofthe template RNA (Ocirc-temp) with each one of Ocirc 5 (see lane 3—Ocirc template+Ocirc 5), Ocirc 6 (see lane 6—Ocirc template+Ocirc6), Ocirc 7 (seelane 8—Ocirctemplate+Ocirc 7), and Ocirc 8 (seelane 11—Ocirc template+Ocirc 8), resulted in slower migrating material indicative ofthe binding of Ocirc 5, 6, 7 and 8, to the template RNA.

A similar experiment, under the same conditions, was performed with Ocirc 2 and 3 as detailed in Table 4 below.

TABLE 4
Preparation of the samples for gel loading (Ocirc 2 and Ocirc 3)

| | | Ocirc-Temp. | Ocirc conc. | Final Conc. |
|---|---|---|---|---|
| sample # | description | conc. μg/μl | μg/μl | (μg/μl) |
| sample #1 | Ocirc-Temp | 0.3811 | — | 0.191 |
| sample #2 | Ocirc-2 | — | 0.2563 | 0.254 |
| sample #3 | Ocirc-3 | — | 0.2531 | 0.257 |
| sample #4 | Ocirc-control | | 0.3881 | |
| sample #5 | Ocirc-Temp + Ocirc-2 | 0.3811 | 0.2563 | 0.191 |
| sample #6 | Ocirc-Temp + Ocirc-3 | 0.3811 | 0.2531 | 0.191 |
| sample #7 | Ocirc-Temp + | 0.3811 | 0.3881 | 0.191 |
| | Ocirc-control | | | |

U2AF2 is a protein that binds the PPT, and splice acceptor sequences and contributes to the splicing event. To test possible binding of U2AF2 protein to the complex of Ocirc+template RNAs, the Ocirc+template RNA oligonucleotides were first hybridized as described above. They were then mixed with the U2AF2 protein (ACRIS) in a binding buffer (final concentration: HEPES-KOH (ph7.6) 20 mM, KCl 100 mM, EDTA 0.2 mM, DTT 0.5 mM). Samples were incubated for 1 hour at 4° C. Preparation for loading on the gel was as described above. Results were inconclusive.

As was discovered for the other Ocirc molecules, mixing of Ocirc 2 (see FIG. 19A lane 7—Ocirc template+Ocirc 2) or Ocirc 3 (see FIG. 19B lane 3—Ocirc template+Ocirc 3) with the template RNA gives rise to slower migrating material indicative of binding to the template RNA. A control Ocirc RNA oligonucleotide, with arms that do not match the template RNA, does not show any binding to the template RNA.

### Example 5: Testing the Ocirc RNA Oligonucleotides in Cells

The set of plasmids described in Example 2 above, namely: pCMV-Rluc-Int-WT, pCMV-Rluc-Int-Mut, pCMV-Rluc-AltInt-WT, pCMV-Rluc-AltInt-Mut were used in the following Example (As used herein, Rluc refers to Renilla luciferase).

As described above, all the plasmids contain the 3′ region of the 3^{rd} intron of the MECP2 gene. In the set of pCMV-Rluc-Int-WT and pCMV-Rluc-Int-Mut the intron was inserted between an AG-GT sequence of the renilla luciferase gene so that the intron starts after the AG and ends before the GT. This insertion site is highly convenient for experimental purposes.

As an alternative, which represents more closely the MECP2 gene in vivo, in the set of pCMV-Rluc-AltInt-WT and pCMV-Rluc-AltInt-Mut the intron was inserted between AG-TCC nucleotides of the renilla luciferase gene. TCC forms the beginning of the 40 exon of the MECP2 gene and is a non-standard and rare exon start.

### Experimental Protocol:

The cell line HEK293 (human embryonic kidney 293) was used in all experiments.

24-well plates were seeded with 100,000 cells per well.

The lipofectamine MessengerMAX Reagent (Invitrogen) was used for transfection as it is suitable for both DNA and RNA. A setup experiment determined that 0.75 μl Lipofectamine and a plasmid concentration of 0.5 μg/well gave the best results.

All protocols were performed according to Manufacturer's instructions.

Cells were collected 48 hours following transfection of plasmid alone or plasmid +oligonucleotide (Ocirc). Cells were lysed in the buffer supplemented in the kit for renilla luciferase assay (Renilla-Glo Luciferase assay system, promega), and renilla luciferase activity was determined according to the Manufacturer's protocol. Reading was done on a luminometer using 96-well plates.

Table 5 shows the results that were obtained for the two plasmid sets.

TABLE 5
Results of the renilla luciferase assay

| Treatment | Average | STDEV |
|---|---|---|
| Untransfected | 6379 | 5593 |
| No Plasmid | 15142 | 2266 |
| pCMV-Rluc-Int_WT 5 1 μg | 6228320 | 1532800 |
| pCMV-Rluc-Int_Mut 8 1 μg | 605617 | 236647 |
| pCMV-Rluc-AltInt_WT 11 1 μg | 6649772 | 244040 |
| pCMV-Rluc-AltInt_Mut 14 1 μg | 64186 | 17580 |
| pCMV-Green Renilla Luc 18 1 μg | 5459719 | 385131 |

While both pCMV-Rluc-Int-WT and pCMV-Rluc-AltInt-WT gave similar results (and ˜20% higher than the starting plasmid pCMV-Green Renilla Luc), the activity of the pCMV-Rluc-Int-Mut plasmid was only 10-fold lower than that of the pCMV-Rluc-Int-WT. In comparison, the activity of the pCMV-Rluc-AltInt-Mut plasmid was 100-fold lower than the WT plasmid. Thus, the pCMV-Rluc-AltInt-WT and pCMV-Rluc-AltInt-Mut set was selected as the target model system for further experiments. This set up can serve for testing the ability of the various Ocirc RNA oligonucleotides in restoring the normal splicing of the MECP2 3^{rd} intron, whereby restoring the normal splicing of the mutated 3^{rd} MECP2 intron by the Ocirc oligonucleotide would be reflected by an increase in the renilla luciferase (Rluc) activity of the pCMV-Rluc-AltInt-Mut plasmid.

### Example 6: Activity of an Ocirc RNA Antisense Oligonucleotide in Cells

To test the activity of the Ocirc RNA oligonucleotides HEK293 cells were grown in 96-well plates-20,000 cells per well.

The plasmids were transfected as described above, using 0.1 μg plasmid per well. The template plasmid was pCMV-RLuc-AltInt-WT. The cells were co-transfected with the template plasmid and with an additional plasmid: either pH1-Ocirc-AS (antisense) or pH1-Ocirc-Control.

The pH1-Ocirc-AS plasmid was constructed by conjugating an Ocirc RNA oligonucleotide, referred to herein as Ocirc-AS (SEQ ID NO: 21) with an H1 promoter.

Ocirc-AS has the following sequence (designated SEQ ID NO: 21):

5′ ACAAACAGAAAGACACAAGGTCTCTGCCTAGCCCCCTTATTCGTCC
TCCCCTTTTCCCTGGGGACTGTGG 3′

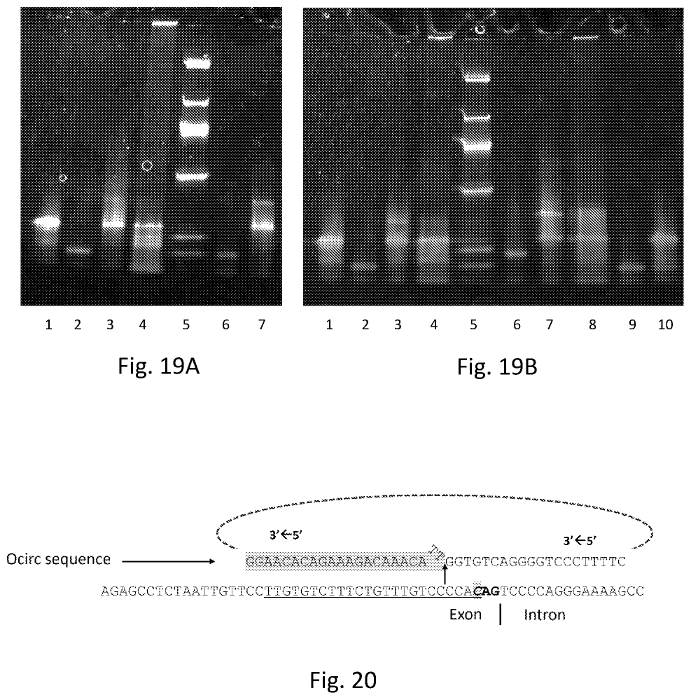

The sequences in bold represent the Ocirc-AS arms which match the target sequence (as shown in FIG. 20), and which cause, upon binding, the encircling of the oligonucleotide. The border between the intron and the 4^{th} exon of MECP2 is indicated. The splice acceptor is shown in bold letters (CAG). The Ocirc sequence is shown on top: the highlighted sequence is the 5′ antisense arm and the non-highlighted sequence is the 3′ antisense arm. RNA produced by H1 promoters ends with TT. Since these TT nucleotides do not match the template RNA they do not bind and thus are illustrated in FIG. 20 as protruding. The dashed line represents the RNA sequence found in between the arms. The black line marks the contact region of the two arms of the Ocirc on the template of the MECP2 sequence. The underlined area is the PPT sequence of the human MECP2 gene.

The pH1-Ocirc-control plasmid was constructed by conjugating a control sequence termed Ocirc-control with an H1 promoter. Ocirc-control has the following sequence (designated SEQ ID NO: 22):

5′ GTGCCGTATGCATCTCTGCCTAGCCCCCTTATTCGTCCTCCCACAA
CTTGCTTG 3′

The sequences in bold represent the Ocirc-control arms which do not match the target sequence and hence this Ocirc-control oligonucleotide should not be able to bind the target.

In both Ocirc-AS and Ocirc-Control the sequences in between the “arms” are identical.

As indicated above these sequences were conjugated with an H1 promoter.

pH1-Ocirc-AS has the following sequence (designated SEQ ID NO: 23):

ACTAGTATATTTGCATGTCGCTATGTGTTCTGGGAAATCACCATAAACG
TGAAATGTCTTTGGATTTGGGAATCTTATAAGTTCTGTATGAGACCACT
CTTTCCCACAAACAGAAAGACACAAGGTCTCTGCCTAGCCCCCTTATTC
GTCCTCCCCTTTTCCCTGGGGACTGTGGTTTTTTGCGGCCGC

pH1-Ocirc-Control has the following sequence (designated SEQ ID NO: 24):

ACTAGTATATTTGCATGTCGCTATGTGTTCTGGGAAATCACCATAAACG
TGAAATGTCTTTGGATTTGGGAATCTTATAAGTTCTGTATGAGACCACT
CTTTCCCGTGCCGTATGCATCTCTGCCTAGCCCCCTTATTCGTCCTCCC
ACAACTTGCTTGTTTTTTGCGGCCGC

The H1 promoter sequence is underlined and the Ocirc sequence is shown in bold. The sequence shown in italics is a striction enzyme site.

The sequence of the plasmid pH1-Ocirc-AS (designated SEQ ID NO: 25) is:

ACTAGTATATTTGCATGTCGCTATGTGTTCTGGGAAATCACCATAAACGTGAAA
TGTCTTTGGATTTGGGAATCTTATAAGTTCTGTATGAGACCACTCTTTCCCACAAACAG
AAAGACACAAGGTCTCTGCCTAGCCCCCTTATTCGTCCTCCCCTTTTCCCTGGGGACTG
TGGTTTTTTGCGGCCGCAAAATCAGCCTCGACTGTGCCTTCTAGTTGCCAGCCATCTGT
TGTTTGCCCCTCCCCCGTGCCTTCCTTGACCCTGGAAGGTGCCACTCCCACTGTCCTTT
CCTAATAAAATGAGGAAATTGCATCACAACACTCAACCCTATCTCGGTCTATTCTTTTG
ATTTATAAGGGATTTTGCCGATTTCGGCCTATTGGTTAAAAAATGAGCTGATTTAACAA
AAATTTAACGCGAATTAATTCTGTGGAATGTGTGTCAGTTAGGGTGTGGAAAGTCCCCA
GGCTCCCCAGCAGGCAGAAGTATGCAAAGCATGCATCTCAATTAGTCAGCAACCAGGTG
TGGAAAGTCCCCAGGCTCCCCAGCAGGCAGAAGTATGCAAAGCATGCATCTCAATTAGT
CAGCAACCATAGTCCCGCCCCTAACTCCGCCCATCCCGCCCCTAACTCCGCCCAGTTCC
GCCCATTCTCCGCCCCATGGCTGACTAATTTTTTTTATTTATGCAGAGGCCGAGGCCGC
CTCTGCCTCTGAGCTATTCCAGAAGTAGTGAGGAGGCTTTTTTGGAGGCCTAGGCTTTT
GCAAAAAGCTCCCGGGAGCTTGTATATCCATTTTCGGATCTGATCAGCACGTGTTGACA
ATTAATCATCGGCATAGTATATCGGCATAGTATAATACGACAAGGTGAGGAACTAAACC
ATGACCGAGTACAAGCCCACGGTGCGCCTCGCCACCCGCGACGACGTCCCCAGGGCCGT
ACGCACCCTCGCCGCCGCGTTCGCCGACTACCCCGCCACGCGCCACACCGTCGATCCGG
ACCGCCACATCGAGCGGGTCACCGAGCTGCAAGAACTCTTCCTCACGCGCGTCGGGCTC
GACATCGGCAAGGTGTGGGTCGCGGACGACGGCGCCGCGGTGGCGGTCTGGACCACGCC
GGAGAGCGTCGAAGCGGGGGCGGTGTTCGCCGAGATCGGCCCGCGCATGGCCGAGTTGA
GCGGTTCCCGGCTGGCCGCGCAGCAACAGATGGAAGGCCTCCTGGCGCCGCACCGGCCC
AAGGAGCCCGCGTGGTTCCTGGCCACCGTCGGCGTCTCGCCCGACCACCAGGGCAAGGG
TCTGGGCAGCGCCGTCGTGCTCCCCGGAGTGGAGGCGGCCGAGCGCGCCGGGGTGCCCG
CCTTCCTGGAGACATCCGCGCCCCGCAACCTCCCCTTCTACGAGCGGCTCGGCTTCACC
GTCACCGCCGACGTCGAGGTGCCCGAAGGACCGCGCACCTGGTGCATGACCCGCAAGCC
CGGTGCCTGACACGTGCTACGAGATTTCGATTCCACCGCCGCCTTCTATGAAAGGTTGG
GCTTCGGAATCGTTTTCCGGGACGCCGGCTGGATGATCCTCCAGCGCGGGGATCTCATG
CTGGAGTTCTTCGCCCACCCCAACTTGTTTATTGCAGCTTATAATGGTTACAAATAAAG
CAATAGCATCACAAATTTCACAAATAAAGCATTTTTTTCACTGCATTCTAGTTGTGGTT
TGTCCAAACTCATCAATGTATCTTATCATGTCTGTATACCGTCGACCTCTAGCTAGAGC
TTGGCGTAATCATGGTCATTACCAATGCTTAATCAGTGAGGCACCTATCTCAGCGATCT
GTCTATTTCGTTCATCCATAGTTGCCTGACTCCCCGTCGTGTAGATAACTACGATACGG
GAGGGCTTACCATCTGGCCCCAGCGCTGCGATGATACCGCGAGAACCACGCTCACCGGC
TCCGGATTTATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGAGTAAGTAGT
TCGCCAGTTAATAGTTTGCGCAACGTTGTTGCCATCGCTACAGGCATCGTGGTGTCACG
CTCGTCGTTTGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATCGTTGTCAGA
AGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACC
GCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCA
ACTGATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCATATTCTT
CCTTTTTCAATATTATTGAAGCATTTATCAGGGTTATTGTCTCATGAGCGGATACATAT
TTGAATGTATTTAGAAAAATAAACAAATAGGGGTCAGTGTTACAACCAATTAACCAATT
CTGAACATTATCGCGAGCCCATTTATACCTGAATATGGCTCATAACACCCCTTGCTCAT
GACCAAAATCCCTTAACGTGAGTTACGCGCGCGTCGTTCCACTGAGCGTCAGACCCCGT
AGAAAAGATCAAAGGATCTTCTTGAGATCCTTTTTTTCTGCGCGTAATCTGCTGCTTGC
AAACAAAAAAACCACCGCTACCAGCGGTGGTTTGTTTGCCGGATCAAGAGCTACCAACT
CTTTTTCCGAAGGTAACTGGCTTCAGCAGAGCGCAGATACCAAATACTGTTCTTCTAGT
GTAGCCGTAGTTAGCCCACCACTTCAAGAACTCTGTAGCACCGCCTACATACCTCGCTC
TGCTAATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGTTCGTG
CACACAGCCCAGCTTGGAGCGAACGACCTACACCGAACTGAGATACCTACAGCGTGAGC
TATGAGAAAGCGCCACGCTTCCCGAAGGGAGAAAGGCGGACAGGTATCCGGTAAGCGGC
AGGGTCGGAACAGGAGAGCGCACGAGGGAGCTTCCAGGGGGAAACGCCTGGTATCTTTA
TAGTCCTGTCGGGTTTCGCCACCTCTGACTTGAGCGTCGATTTTTGTGATGCTCGTCAG
GGGGGCGGAGCCTATGGAAAAACGCCAGCAACGCGGCCTTTTTACGGTTCCTGGCCTTT
TGCTGGCCTTTTGCTCACATGTTCTTTCCTGCGTTATCCCCTGATTCTGTGGATAACCG
TATTACCGCCTTTGAGTGAGCTGATACCGCTCGCCGCAGCCGAACGACCGAGCGCAGCG
AGTCAGTGAGCGAGGAAGCGGAAGGCGAGAGTAGGGAACTGCCAGGCATCAAACTAAGC
AGAAGGCCCCTGACGGATGGCCTTTTTGCGTTTCTACAAACTCTTTCTGTGTTGTAAAA
CGACGGCCAGTCTTAAGCTCGGGCCCCCTGGGCGGTTCTGATAACGAGTAATCGTTAAT
CCGCAAATAACGTAAAAACCCGCTTCGGCGGGTTTTTTTATGGGGGGAGTTTAGGGAAA
GAGCATTTGTCAGAATATTTAAGGGCGCCTGTCACTTTGCTTGATATATGAGAATTATT
TAACCTTATAAATGAGAAAAAAGCAACGCACTTTAAATAAGATACGTTGCTTTTTCGAT
TGATGAACACCTATAATTAAACTATTCATCTATTATTTATGATTTTTTGTATATACAAT
ATTTCTAGTTTGTTAAAGAGAATTAAGAAAATAAATCTCGAAAATAATAAAGGGAAAAT
CAGTTTTTGATATCAAAATTATACATGTCAACGATAATACAAAATATAATACAAACTAT
AAGATGTTATCAGTATTTATTATCATTTAGAATAAATTTTGTGTCGCCCTTAATTGTGA
GCGGATAACAATTACGAGCTTCATGCACAGTGGCGTTGACATTGATTATTGACTAGCAT
GTTCTTTCCTGCGTTATCCCCTGATTCTGTGGATAACCGTATTACCGCCATGCATTAGT
TATTAATAACATACGCTCTCCATCAAAACAAAACGAAACAAAACAAACTAGCAAAATAG
GCTGTCCCCAGTGCAAGTGCAGGTGCCAGAACATTTCTCT

The sequence of the plasmid pH1-Ocirc-Control (designated SEQ ID NO: 26) is:

ACTAGTATATTTGCATGTCGCTATGTGTTCTGGGAAATCACCATAAACGTGAAA
TGTCTTTGGATTTGGGAATCTTATAAGTTCTGTATGAGACCACTCTTTCCCGTGCCGTA
TGCATCTCTGCCTAGCCCCCTTATTCGTCCTCCCACAACTTGCTTGTTTTTTGCGGCCG
CAAAATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGGA
AATTGCATCACAACACTCAACCCTATCTCGGTCTATTCTTTTGATTTATAAGGGATTTT
GCCGATTTCGGCCTATTGGTTAAAAAATGAGCTGATTTAACAAAAATTTAACGCGAATT
AATTCTGTGGAATGTGTGTCAGTTAGGGTGTGGAAAGTCCCCAGGCTCCCCAGCAGGCA
GAAGTATGCAAAGCATGCATCTCAATTAGTCAGCAACCAGGTGTGGAAAGTCCCCAGGC
TCCCCAGCAGGCAGAAGTATGCAAAGCATGCATCTCAATTAGTCAGCAACCATAGTCCC
GCCCCTAACTCCGCCCATCCCGCCCCTAACTCCGCCCAGTTCCGCCCATTCTCCGCCCC
ATGGCTGACTAATTTTTTTTATTTATGCAGAGGCCGAGGCCGCCTCTGCCTCTGAGCTA
TTCCAGAAGTAGTGAGGAGGCTTTTTTGGAGGCCTAGGCTTTTGCAAAAAGCTCCCGGG
AGCTTGTATATCCATTTTCGGATCTGATCAGCACGTGTTGACAATTAATCATCGGCATA
GTATATCGGCATAGTATAATACGACAAGGTGAGGAACTAAACCATGACCGAGTACAAGC
CCACGGTGCGCCTCGCCACCCGCGACGACGTCCCCAGGGCCGTACGCACCCTCGCCGCC
GCGTTCGCCGACTACCCCGCCACGCGCCACACCGTCGATCCGGACCGCCACATCGAGCG
GGTCACCGAGCTGCAAGAACTCTTCCTCACGCGCGTCGGGCTCGACATCGGCAAGGTGT
GGGTCGCGGACGACGGCGCCGCGGTGGCGGTCTGGACCACGCCGGAGAGCGTCGAAGCG
GGGGCGGTGTTCGCCGAGATCGGCCCGCGCATGGCCGAGTTGAGCGGTTCCCGGCTGGC
CGCGCAGCAACAGATGGAAGGCCTCCTGGCGCCGCACCGGCCCAAGGAGCCCGCGTGGT
TCCTGGCCACCGTCGGCGTCTCGCCCGACCACCAGGGCAAGGGTCTGGGCAGCGCCGTC
GTGCTCCCCGGAGTGGAGGCGGCCGAGCGCGCCGGGGTGCCCGCCTTCCTGGAGACATC
CGCGCCCCGCAACCTCCCCTTCTACGAGCGGCTCGGCTTCACCGTCACCGCCGACGTCG
AGGTGCCCGAAGGACCGCGCACCTGGTGCATGACCCGCAAGCCCGGTGCCTGACACGTG
CTACGAGATTTCGATTCCACCGCCGCCTTCTATGAAAGGTTGGGCTTCGGAATCGTTTT
CCGGGACGCCGGCTGGATGATCCTCCAGCGCGGGGATCTCATGCTGGAGTTCTTCGCCC
ACCCCAACTTGTTTATTGCAGCTTATAATGGTTACAAATAAAGCAATAGCATCACAAAT
TTCACAAATAAAGCATTTTTTTCACTGCATTCTAGTTGTGGTTTGTCCAAACTCATCAA
TGTATCTTATCATGTCTGTATACCGTCGACCTCTAGCTAGAGCTTGGCGTAATCATGGT
CATTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTG
GCCCCAGCGCTGCGATGATACCGCGAGAACCACGCTCACCGGCTCCGGATTTATCAGCA
ATAAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGTT
TGCGCAACGTTGTTGCCATCGCTACAGGCATCGTGGTGTCACGCTCGTCGTTTGGTATG
GCTTCATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTA
AGATGCTTTTCTGTGACTGGTGAGTACTCAACCAAGTCATTCTGAGAATAGTGTATGCG
GCGACCGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAA
AGGGAATAAGGGCGACACGGAAATGTTGAATACTCATATTCTTCCTTTTTCAATATTAT
TGAAGCATTTATCAGGGTTATTGTCTCATGAGCGGATACATATTTGAATGTATTTAGAA
AAATAAACAAATAGGGGTCAGTGTTACAACCAATTAACCAATTCTGAACATTATCGCGA
GCCCATTTATACCTGAATATGGCTCATAACACCCCTTGCTCATGACCAAAATCCCTTAA
CGTGAGTTACGCGCGCGTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAGGTAA
CTGGCTTCAGCAGAGCGCAGATACCAAATACTGTTCTTCTAGTGTAGCCGTAGTTAGCC
CACCACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACGATAGT
TACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCTGTCGGGTTT
CGCCACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGCTC
ACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCAGTGAGCGAGGA
AGCGGAAGGCGAGAGTAGGGAACTGCCAGGCATCAAACTAAGCAGAAGGCCCCTGACGG
ATGGCCTTTTTGCGTTTCTACAAACTCTTTCTGTGTTGTAAAACGACGGCCAGTCTTAA
GCTCGGGCCCCCTGGGCGGTTCTGATAACGAGTAATCGTTAATCCGCAAATAACGTAAA
AACCCGCTTCGGCGGGTTTTTTTATGGGGGGAGTTTAGGGAAAGAGCATTTGTCAGAAT
ATTTAAGGGCGCCTGTCACTTTGCTTGATATATGAGAATTATTTAACCTTATAAATGAG
AAAAAAGCAACGCACTTTAAATAAGATACGTTGCTTTTTCGATTGATGAACACCTATAA
TTAAACTATTCATCTATTATTTATGATTTTTTGTATATACAATATTTCTAGTTTGTTAA
AGAGAATTAAGAAAATAAATCTCGAAAATAATAAAGGGAAAATCAGTTTTTGATATCAA
AATTATACATGTCAACGATAATACAAAATATAATACAAACTATAAGATGTTATCAGTAT
TTATTATCATTTAGAATAAATTTTGTGTCGCCCTTAATTGTGAGCGGATAACAATTACG
AGCTTCATGCACAGTGGCGTTGACATTGATTATTGACTAGCATGTTCTTTCCTGCGTTA
TCCCCTGATTCTGTGGATAACCGTATTACCGCCATGCATTAGTTATTAATAACATACGC
TCTCCATCAAAACAAAACGAAACAAAACAAACTAGCAAAATAGGCTGTCCCCAGTGCAA
GTGCAGGTGCCAGAACATTTCTCT

48 hours after transfections, the cells were harvested and subjected to a Renilla luciferase assay analysis. All experiments were done in quadruplets.

### Results

| | | | Standard |
|---|---|---|---|
| Template Plasmid | Test plasmid | Rluc activity | deviation |
| pCMV-RLuc-AltInt- | — | 5,047,058 | 1,351,618 |
| WT | | | |
| pCMV-RLuc-AltInt- | pH 1-Ocirc-Control | 4,873,062 | 907,102 |
| WT | | | |
| pCMV-RLuc-AltInt- | pH 1-Ocirc-AS | 3,258,109 | 935,339 |
| WT | | | |

The results of the experiment were modified by removing extreme points, and showed the following:

| | | | Standard |
|---|---|---|---|
| Template Plasmid | Test plasmid | Rluc activity | deviation |
| pCMV-RLuc-AltInt- | — | 4,779,521 | 740,067 |
| WT | | | |
| pCMV-RLuc-AltInt- | pH 1-Ocirc-Control | 6,467,840 | 907,102 |
| WT | | | |
| pCMV-RLuc-AltInt- | pH 1-Ocirc-AS | 3,671,592 | 535,237 |
| WT | | | |

A decrease of-33% to 43% in Renilla luciferase activity was observed following addition of the pH1-Ocirc-AS plasmid that expresses the Ocirc-AS RNA, as compared to the parallel transfection with the pH1-Ocirc-Control plasmid that expresses the Ocirc-control RNA.

These results show that a plasmid containing Ocirc RNA in capable of entering the cell nucleus, binding to its target sequence and successfully decreasing the expression of a target gene, on the RNA level.

1. An oligonucleotide, comprising from 5′ to 3′:
A first sequence of nucleic acids that is complementary in its 3′ to 5′ direction to a region in a pre-mRNA or mRNA target molecule;
A second sequence of nucleic acids comprising a heterologous sequence; and
a third sequence of nucleic acids that is complementary in its 3′ to 5′ direction to a sequence of nucleic acids in the pre mRNA or mRNA target molecule which is positioned upstream to the hybridization site of the first sequence of nucleic acids;
and wherein said first and third sequences of nucleic acids hybridize to the same intron, or to the same exon, or to successive intron and exon, or to successive exon and intron in the target molecule.

2. The oligonucleotide of claim 1 wherein said heterologous sequence comprises a sequence which is identical to and is in the same 5′->3′ direction as the sequence of an exon, an intron, a splice site, a 5′ UTR, a 3′ UTR, or a fragment or portion thereof of the wildtype pre-mRNA or mRNA target molecules.

3. The oligonucleotide of claim 1 wherein said heterologous sequence encodes a portion of an exon.

4. The oligonucleotide of claim 1 wherein said oligonucleotide is an antisense oligonucleotide.

5. The oligonucleotide of claim 1, wherein said oligonucleotide is synthesized as a linear single stranded molecule and forms an open circle structure upon hybridization with the pre-mRNA target molecule.

6. The oligonucleotide of claim 1 wherein hybridization of the oligonucleotide with the target pre-mRNA or mRNA molecule masks a mutation in the pre-mRNA or mRNA molecule and aligns the second sequence of nucleic acids such that the mutated sequence of the pre-mRNA is replaced with the sequence of the wildtype pre-mRNA, thereby allowing the translation of a functional protein.

7. The oligonucleotide of claim 1 wherein hybridization of the oligonucleotide with the target pre-mRNA or mRNA molecule introduces to the endogenous pre-mRNA or mRNA molecules a heterologous motif.

8. The oligonucleotide of claim 1, wherein said second sequence of nucleic acids binds to a cellular complex.

9. The oligonucleotide of claim 1 wherein said nucleic acids are ribonucleotides.

10. The oligonucleotide of claim 1 wherein said mutated site comprises a single base mutation, a substitution, a deletion mutation, an insertion mutation, or an InDel mutation.

11. The oligonucleotide of claim 1 wherein said second sequence of nucleic acids comprises (i) a portion of an intron ending with an acceptor site; (ii) a heterologous sequence to be trans-spliced into the target pre-mRNA molecule; (iii) a portion of an intron comprising a donor site, and optionally a branch point and a PPT sequence, ending at the proximity of an acceptor site sequence in the wildtype exon.

12. An oligonucleotide, comprising from 5′ to 3′:
a first sequence of nucleic acids that is complementary in its 3′ to 5′ direction to a region in a pre-mRNA target molecule;
a second sequence comprising:
(i) a portion of an intron ending with an acceptor site;
(ii) a heterologous sequence to be trans-spliced into the target pre-mRNA molecule; and
(iii) a portion of an intron comprising a donor site, and optionally a branch point and a PPT sequence, ending at the proximity of an acceptor site sequence in the wildtype exon, and
a third sequence of nucleic acids that hybridizes in its 3′ to 5′ direction with a sequence of nucleic acids positioned upstream to the hybridization site of the first sequence of nucleic acids in said pre-mRNA target molecule preceding said full or partial acceptor site sequence,
wherein said first and third sequences of nucleic acids hybridize to the same intron, or to the same exon, or to successive intron and exon, or to successive exon and intron.

13. The oligonucleotide of claim 12 wherein said heterologous sequence comprises a sequence which is identical to and is in the same 5′->3′ direction as the sequence of an exon, an intron, a splice site, or a fragment or portion thereof of the wildtype pre-mRNA molecule terminating in the YAG acceptor site following the second complementary sequence at the 3′ terminus of the oligonucleotide.

14. The oligonucleotide of claim 12 wherein said heterologous sequence encodes a portion of an exon.

15. The oligonucleotide of claim 1, wherein said oligonucleotide is selected from a group consisting of Ocirc 1 (SEQ ID NO: 12), Ocirc 2 (SEQ ID NO: 13), Ocirc 3 (SEQ ID NO: 14), Ocirc 4 (SEQ ID NO: 15), Ocirc 5 (SEQ ID NO: 16), Ocirc 6 (SEQ ID NO: 17), Ocirc 7 (SEQ ID NO: 18), and Ocirc 8 (SEQ ID NO: 19).

16. A delivery vector comprising the oligonucleotide of claim 1.

17. An isolated cell comprising the oligonucleotide of claim 1.

18. A method for substitution of an endogenous nucleic acid sequence comprising bringing into contact the oligonucleotides of claim 1 with a target cell comprising said endogenous nucleic acid sequence.

19. A method of treating Rett syndrome, said method comprises administering the oligonucleotide of claim 1 to a patient in need thereof.

20. (canceled)
